(19) United States
(12) Patent Application Publication
(10) Pub. No.: US 2022/0153780 A1
(43) Pub. Date: May 19, 2022

(54) SALT FORMS OF S-(N, N-DIETHYLCARBAMOYL)GLUTATHIONE

(71) Applicant: Tonix Pharma Holdings Limited, Dublin 2 (IE)

(72) Inventors: Gregory M. Sullivan, Barryville, NY (US); Siobhan J. Fogarty, Dublin (IE)

(21) Appl. No.: 17/442,258

(22) PCT Filed: Mar. 26, 2020
(86) PCT No.: PCT/IB2020/000237

§ 371 Date: Sep. 23, 2021

Related U.S. Application Data
(60) Provisional application No. 62/941,533, filed on Nov. 27, 2019.
(60) Provisional application No. 62/824,008, filed on Mar. 26, 2019.

(51) Int. Cl. C07K 5/02

(52) U.S. Cl. CPC C07K 5/0215; A61K 38/00; C07B 2200/13

(57) ABSTRACT

The invention relates in various aspects to a salt form S—(N, N-diethylcarbamoyl)glutathione, a method of producing the salt form, a pharmaceutical composition comprising said salt form. The invention also relates to a method of preventing or treating a glutamate-related disorder comprising administering to said subject a therapeutically effective amount of said salt form.

### BACKGROUND OF THE DISCLOSURE

Alcohol Use Disorder (AUD) is a complex and devastating disease, affecting 13.9% of Americans in a 1-year period and resulting in a range of medical, psychological, social, economic, and personal problems. Problem drinking costs the U.S. society more than $249 billion annually and causes nearly 88,000 deaths each year (Centers for Disease Control and Prevention, 2013). Advances have been made in developing effective treatments for AUD, especially medications. Specifically, four medications are approved for alcohol dependence by the U.S. Food and Drug Administration (FDA): Disulfiram, oral Naltrexone, long-acting injectable Naltrexone, and Acamprosate. In addition, Nalmefene was approved in Europe by the European Medicines Agency for the treatment of alcohol dependence.

Several factors contribute to the development of AUDs. Their heterogeneity presents challenges for developing broadly effective pharmacotherapeutic interventions. Current evidence supports the roles of several neurotransmitter systems in the neurobiological dysfunction associated with AUDs. These include mesolimbic dopaminergic mechanisms, abnormalities in serotonergic, gamma-aminobutyric acid (GABA)-ergic, and glutamatergic neurotransmission, as well as the role of proopiomelanocortin (POMC) peptides such as the endogenous opioids. Additionally, a number of other neurotransmitters important in the stress response system have also been implicated.

Disulfiram (DSF) is an aldehyde dehydrogenase (ALDH) inhibitor which has been employed for the treatment of alcohol (ethanol) abuse and alcoholism for over 65 years (Hald and Jacobson. 1948. Lancet 2, 1001-04). DSF's inhibition of hepatic mitochondrial ALDH₂ blocks the second step in alcohol metabolism. Thus, any subsequent consumption of ethanol results in an accumulation of the toxic intermediate, acetaldehyde. This produces the adverse effect known as the disulfiram-ethanol reaction (DER) when ethanol is consumed by patients being treated with DSF. Specifically, acetaldehyde accumulation results in a potent systemic vasodilatory response with symptoms such as flushing, headache, nausea, and tachycardia.

Naltrexone, sold under the brand names ReVia and Vivitrol, is a competitive antagonist of opioid receptors and Acamprosate, sold under the brand name Campral, is a medication which is believed to act as an NMDA receptor antagonist and positive allosteric modulator of GABA receptors.

AUD consists of multiple neurobiological mechanisms and through complex genetic and environmental interactions, exhibits a variety of phenotypes. Because of this heterogeneity, no medication works for everyone and in every situation. Thus, there exists a need to discover and develop new, more effective, bioavailable and well-tolerated medications to deter ethanol consumption by humans and to treat glutamate-related disorders, while concurrently avoiding the adverse side effects associated with ALDH₂ inhibition and the DERs associated therewith.

### SUMMARY OF THE DISCLOSURE

The disclosure, in one aspect, is based on the finding that a salt form of S—(N, N-diethylcarbamoyl)glutathione (carbamathione) improves carbamathione solubility and other physiochemical properties of carbamathione.

Therefore, in a first aspect, the disclosure relates to a salt form of S—(N, N-diethylcarbamoyl)glutathione, wherein the salt is selected from the group consisting of an acetate salt, an adipate salt, an ascorbate salt, a benzoate salt, a camphorate salt, a citrate salt, a fumarate salt, a glutarate salt, a glycolate salt, a hydrochloride salt, a tartrate salt, a malate salt, a maleate salt, a methanesulfonate salt, an ethanedisulfonate salt, an ethanesulfonate salt, a naphthalenesulfonate salt, an oxalate salt, a phosphate salt, a sulfate salt, a sorbate salt, a benzenesulfonate, a cyclamate salt, succinate salt, a toluenesulfonate salt, an arginine salt, a lysine salt, a deanol salt, a choline salt, a sodium salt, a potassium salt, a diethylammonium salt, a meglumine salt, a pyridoxine salt, a tris(hydroxymethyl)ammonium salt, an N-cyclohexylsulfamate salt, camphor-10-sulfonate salt, a naphthalenedisulfonate salt, and a quinaldate salt its solvates, polymorphs, hydrates or mixtures thereof.

In another aspect, the disclosure relates to a pharmaceutical composition comprising: (i) a therapeutically effective amount of a salt form according to the first aspect of the invention, wherein the salt form is crystalline, co-crystalline, semi-crystalline or amorphous, or its solvates, polymorphs, hydrates or mixtures thereof; and (ii) at least one pharmaceutically acceptable carrier.

In another aspect, the disclosure relates to a pharmaceutical composition comprising: (i) 30 mg to 4000 mg of a salt form according to the first aspect of the invention, wherein the salt form is crystalline, co-crystalline, semi-crystalline or amorphous, or its solvates, polymorphs, hydrates or mixtures thereof; and (ii) at least one pharmaceutically acceptable carrier.

In another aspect, the disclosure relates to a method of preventing or treating a glutamate-related disorder in a subject in need thereof or at risk of, comprising administering to said subject a therapeutically effective amount of a salt form according to the first aspect of the invention or a pharmaceutical composition according to another aspect of this disclosure.

### BRIEF DESCRIPTION OF THE DRAWINGS

The foregoing summary, as well as the following detailed description of the disclosure, will be better understood when read in conjunction with the appended drawings. For the purpose of illustrating the disclosure there are shown in the drawings embodiment(s) which are presently preferred. It should be understood, however, that the disclosure is not limited to the precise arrangements and instrumentalities shown.

FIG. 1 shows the effects of intraperitoneal administration of carbamathione (0, 100, 200, 400 mg/kg) on 2-hour ethanol intake (g/kg) by adult male P rats.

FIG. 2 shows the effects of intraperitoneal administration of carbamathione (0, 100, 200, 400 mg/kg) on 2-hour ethanol intake (g/kg) by adult male HAD1 rats.

FIG. 3 is a graph showing weekly average ethanol intake (g/kg) comparing a group of mice exposed to chronic intermittent ethanol (CIE) vapor exposure in inhalation chambers and another group of mice (CTL) treated similarly but exposed to air in inhalation chambers. All mice received intraperitoneal administration (IP) of saline solution prior to the start of daily drinking sessions during Baseline and the early Test cycles to acclimate the animals to the handling procedure.

FIG. 4 is a graph showing weekly average ethanol intake (g/kg) comparing CIE and CTL mice, which received IP injections of carbamathione (100, 200, or 400 mg/kg) or vehicle (0.25% CMC in water) 30 min before drinking.

FIG. 5 is a graph showing weekly average ethanol intake (g/kg) comparing CIE and CTL mice treated with 400 mg/kg carbamathione and exposed to a sixth cycle of CIE (Test 6).

FIG. 6 is a graph showing weekly average ethanol intake (g/kg), where mice that received 100 or 200 mg/kg carbamathione were combined and randomly redistributed to receive 75 or 100 mg/kg disulfiram during the first two days and these doses were increased to 125 and 150 mg/kg disulfiram, respectively for the last three days of Test 6.

FIG. 7 is a graph showing weekly average ethanol intake (g/kg) of mice treated with 125 and 150 mg/kg disulfiram.

FIG. 8 is a graph showing weekly average ethanol intake (g/kg) after a seventh CIE or air exposure cycle and a 600 mg/kg carbamathione dose.

FIG. 9 is a graph showing the results obtained during Test cycles 5 and 7 expressed as percent change from the corresponding CIE or CTL vehicle-injected group for mice that received treatment with 100, 200, 400, or 600 mg/kg doses of carbamathione.

FIG. 10 is the ¹H-NMR spectrum (D₂O, 400 MHz) of carbamathione (TNX1001-SM).

FIG. 11 is the XRPD pattern of carbamathione (TNX1001-SM).

FIG. 12 is the DSC profile of TNX1001-SM.

FIG. 13 is the TGA (black line) and dTGA (red line) of TNX1001-SM.

FIG. 14 is the FT-IR spectrum of TNX1001-SM.

FIG. 15 is the XRPD pattern of the solid sample collected from the high temperature evaporation of water experiment with the co-former L-lysine (“LLYS”) (top). The diffractogram of TNX1001-LLYS-NP01 (bottom) is reported as reference.

FIG. 16 is a XRPD pattern comparison between the sample recovered from the high temperature evaporation of water experiments and the same sample after 1 day (middle) and 4 days (top).

FIG. 17 is the XRPD pattern of the solid sample collected from the slurry experiment in water with the co-former NaOH (middle). The signal at 2θ 18° was due to residual material from the vial cap.

FIG. 18 is the XRPD pattern of the solid sample collected in methanol slurry experiment with L-Lys (second from top). The diffractograms of TNX1001-SM (second from bottom), L-Lysine (bottom) and TNX1001-LLYS-NP02 (top) are reported as reference standard.

FIG. 19 is the XRPD pattern of the solid sample collected in the methanol slurry experiment with L-Lys after drying (top). The diffractograms of TNX1001-LLYS-NP01 (middle) and TNX1001-LLYS-NP02 (bottom) are also reported as reference.

FIG. 20 is the XRPD pattern of sample TNX1001-LLYS-SL-MET-dried after storage under ambient conditions for 24 hours (middle) compared to the XRPD pattern of the same sample acquired before the storage (top). The diffractogram of TNX1001-LLYS-NP02 (bottom) is reported as reference

FIG. 21 is the XRPD pattern of the solid sample collected from the DCM slurry experiment with L-Lysine (top). The diffractogram of TNX1001-LLYS-NP02 is reported as reference (bottom).

FIG. 22 is the XRPD pattern of the solid samples collected in the DCM slurry experiment with p-toluenesulfonic acid.

FIG. 23 is the XRPD patterns of solid samples collected from the kneading experiments with a catalytic amount of H₂O with the co-former L-lysine (top). The diffractograms of TNX1001-LLYS-NP02 (bottom) is reported as a reference.

FIG. 24 is the XRPD patterns of solid samples collected from the kneading experiments with a catalytic amount of H₂O with the co-formers sulfuric acid (top) and methanesulfonic acid (bottom).

FIG. 25 is the XRPD pattern of solid sample recovered from the experiment with hydrochloric acid as a co-former.

FIG. 26 is the XRPD pattern comparison between the sample recovered from high temperature evaporation of aqueous solution of TNX1001-SM and L-lysine (top) and the reference standard TNX1001-LLYS-NP01.

FIG. 27 is the XRPD pattern of TNX1001-LLYS-NP01.

FIG. 28 is the DSC profile of TNX1001-LLYS-NP01.

FIG. 29 is the TGA (solid line) and the dTGA (dotted line) of the sample TNX1001-LLYS-NP01.

FIG. 30 is the FT-IR spectrum of sample TNX1001-LLYS-NP01.

FIG. 31 is the comparison of the FT-IR spectrum of sample TNX1001-LLYS-NP01 (bottom) with the TNX1001-SM reference (middle) and L-lysine (top).

FIG. 32 is an enlargement of FIG. 31 between 2200-600 cm⁻¹.

FIG. 33 is the ¹H-NMR spectrum (D₂O, 400 MHz) of TNX1001-LLYS-NP01.

FIG. 34 is the XRPD pattern of TNX1001-LLYS-NP02.

FIG. 35 is the DVS isotherm plot of the carbamathione lysine salt (TNX1001-LLYS-NP01).

FIG. 36 is a plot depicting the DVS change in mass vs time for the carbamathione lysine salt during DVS analysis.

FIG. 37 is a plot depicting the solubility of TNX1001-LLYS-NP01 vs temperature (° C.) in a solution having a pH of 6.8. The circles correspond to the observed solubility of TNX1001-LLYS-NP01; the square corresponds to the estimated solubility of TNX1001-LLYS-NP01 at 25° C.; and the diamond corresponds to the solubility of free carbamathione (TNX1001) at 25° C. at pH 6.8.

FIG. 38 is a plot depicting the solubility of TNX1001-LLYS-NPO1 vs temperature (° C.) in a solution having a pH of 4.5. The circles correspond to the observed solubility of TNX1001-LLYS-NP01; the square corresponds to the estimated solubility of TNX1001-LLYS-NP01 at 25° C.; and the diamond corresponds to the solubility of free carbamathione (TNX1001) at 25° C. at pH 4.5.

### DETAILED DESCRIPTION OF THE DISCLOSURE

### General Techniques

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well-known and commonly used in the art. In case of conflict, the present specification, including definitions, will control.

Throughout this specification and embodiments, the word “comprise,” or variations such as “comprises” or “comprising,” will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a”, “an” and “the” are used herein to refer to one or to more than one (i.e., to at least one) of the grammatical object of the article. By way of example, “an element” means one element or more than one element. Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Exemplary methods and materials are described herein, although methods and materials similar or equivalent to those described herein can also be used in the practice or testing of the present application. The materials, methods, and examples are illustrative only and not intended to be limiting.

### Definitions

The following terms, unless otherwise indicated, shall be understood to have the following meanings:

As used herein, the terms “free carbamathione”, “parent carbamathione”, “free S—(N,N-diethylcarbamoyl)glutathione” and “parent S—(N,N-diethylcarbamoyl)glutathione” are used interchangeably, and refer to carbamathione (i.e., S—(N, N-diethylcarbamoyl)glutathione) in its neutral form, i.e., unreacted with acidic or basic co-formers.

As used herein, the term “solvate” refers to an aggregate that consists of a solute ion or molecule with one or more solvent molecules such as with water (also known as hydrates), methanol, ethanol, dimethylformamide, diethyl ether, acetamide, and the like. Mixtures of such solvates can also be prepared. Solvation involves different types of intermolecular interactions: hydrogen bonding, ion-dipole interactions, and van der Waals forces (which consist of dipole-dipole, dipole-induced dipole, and induced dipole-induced dipole interactions). The source of such solvates can be from the solvent of crystallization, inherent in the solvent of preparation or crystallization, or adventitious to such solvent.

As used herein, the term “polymorph” refers to different crystalline forms of the same compound and other solid-state molecular forms including co-crystals, semi-crystals, amorphous powders, pseudo-polymorphs, such as hydrates, solvates, or salts of the same compound. Different crystalline polymorphs have different crystal structures due to a different packing of molecules in the lattice, as a result of changes in temperature, pressure, or variations in the crystallization process. Polymorphs differ from each other in their physical properties, such as X-ray diffraction characteristics, stability, melting points, solubility, or rates of dissolution in certain solvents. Thus, crystalline, polymorphic forms are important aspects in the development of suitable dosage forms in pharmaceutical industry.

As used herein, the term “hydrate” refers to a compound, typically a crystalline one, in which water molecules are chemically bound to another compound or an element. Hydrates may also refer to compositions wherein water has been incorporated into the crystalline structure without chemical alteration to the other compound. Hydrates may include monohydrates, dihydrates, trihydrates, tetrahydrates, and so on.

As used herein, the term “metabolite” is intended to encompass compounds that are produced by metabolism/biochemical modification of the parent compound under physiological conditions, e.g. through certain enzymatic pathways.

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the pharmaceutical composition is administered. An “excipient”, as used herein, refers to a non-toxic material that does not interfere with the activity of the active ingredient. Examples of suitable pharmaceutical carriers are described in “Remington's Pharmaceutical Sciences” by E. W. Martin. The formulation should suit the mode of administration.

The term “pharmaceutically acceptable salt” refers to salts which retain the biological effectiveness and properties of the compounds of this disclosure and which are not biologically or otherwise undesirable. In some embodiments, the compounds of this disclosure are capable of forming acid and/or base salts by virtue of the presence of amino, and/or carboxylic acid groups or groups similar thereto. Pharmaceutically acceptable acid addition salt forms can be prepared from inorganic and organic acids. Pharmaceutically acceptable base addition salts can be prepared from inorganic and organic bases.

The terms “patient”, “subject”, or “individual” are used interchangeably herein and refer to either a human or a non-human animal. These terms include mammals, such as humans, primates, livestock animals (including bovines, porcines, camels, etc.), companion animals (e.g., canines, felines, etc.) and rodents (e.g., mice and rats).

As used herein, the terms “prevent”, “preventing” and “prevention” refer to the prevention of the recurrence or onset of, or a reduction in one or more symptoms of a disease (e.g., a glutamate-related disorder) in a subject as a result of the administration of a therapy in an initial or early stage of the disease (e.g., a prophylactic or therapeutic agent). For example, in the context of the administration of a therapy to a subject for a disorder, “prevent”, “preventing” and “prevention” refer to the inhibition or a reduction in the development or onset of the disorder, or the prevention of the recurrence, onset, or development of one or more symptoms of the disorder, in a subject resulting from the administration of a therapy (e.g., a prophylactic or therapeutic agent), or the administration of a combination of therapies (e.g., a combination of prophylactic or therapeutic agents).

As used herein, the terms “treat”, “treating” or “treatment” is used to designate the administration of the compound to control the progression of the disease after the clinical signs have appeared. Control of the progression of the disease is understood as the beneficial or desired clinical results which include, but are not limited to, reduction of the symptoms, reduction of the duration of the disease, stabilization of pathological conditions (specifically avoiding additional impairment), delaying the progression of the disease, improving the pathological condition and remission (both partial and complete).

“Administering” or “administration of” a substance, a compound or an agent to a subject can be carried out using one of a variety of methods known to those skilled in the art. For example, a compound or an agent can be administered orally, sublingually, intranasally, transdermally, subcutaneously, intramuscularly, intraperitoneally, intravenously, conjunctival, intrathecally, by inhalation into the lung or rectally. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

The term “glutamate related disorder” includes, but is not limited to, neurodegenerative diseases associated with elevated levels of extracellular glutamate, including, but not limited to, Huntington's disease, Alzheimer's disease, Parkinson's disease, acquired immunodeficiency syndrome (AIDS) neuropathy, epilepsy, nicotine addiction, cerebral ischemia (stroke), and familial Amyotrophic Lateral Sclerosis (ALS); as well as neurodegenerative diseases associated with thiamine deficiency, such as Wemicke-Korsakoff syndrome, cerebral beriberi, Machado-Joseph disease, Soshin disease, and related diseases. Glutamate-related diseases also include diseases or conditions wherein glutamate related activity is implicated, such as anxiety, glutamate related convulsions, hepatic encephalopathy, neuropathic pain, domoic acid poisoning, hypoxia, anoxia, mechanical trauma to the nervous system, hypertension, alcohol withdrawal seizures, alcohol addiction, alcohol craving, cardiovascular ischemia, oxygen convulsions, and hypoglycemia. Other disorders which have been linked to excess or aberrant activation of glutamate receptors include Creutzfeldt-Jakob disease (Muller et al., Mech. Ageing. Dev., 116:193 (2000)), nicotine addiction, cocaine addiction (Ciano & Everitt, Neuropsychopharmacology, 25:341 (2001)), noise induced hearing loss (Chen et al., Hear. Res., 154: 108 (2001), heroin addiction and addiction to other opioids (Bisaga et al., Psychopharmacology (Bert), 157: 1 (2001)), cyanide-induced apoptosis (Jensen et al., Toxicol. Sci., 58:127 (2000)), schizophrenia (Bird et al., Psychopharmacology (Bert), 155:299 (2001)), bipolar disorder (Dean et al., J. Affect. Disord, 66:147 (2001)), peripheral neuropathy associated with diabetes (Elgado-Esteban et al., J. Neurochem, 75:1618 (2000)), gambling disorder, mood symptoms relating to addiction withdrawal and non-ketonic hyperglycinemia (Deutsch et al., Clin. Neuropharmacol., 21:71 (1998)).

As used herein, the term “area under the curve” or “AUC” is the definite integral in a plot of drug concentration in blood plasma vs. time. The AUC reflects the actual body exposure to drug after administration of a dose of the drug and is expressed in h μg/mL. The area under the curve is dependent on the rate of elimination of the drug from the body and the dose administered. The total amount of drug eliminated by the body may be assessed by adding up or integrating the amounts eliminated in each time interval, from time zero (time of the administration of the drug) to infinite time. This total amount corresponds to the fraction of the dose administered that reaches the systemic circulation.

### Salt Forms of S—(N, N-diethylcarbamoyl)glutathione

In one aspect, the invention provides a composition comprising a salt form of S—(N,N-diethylcarbamoyl)glutathione with improved solubility, enhanced physiochemical properties, bioavailability, absorption, stability and/or other more favorable properties, as compared to the neutral parent compound.

In one aspect, the disclosure relates to a salt form of S—(N,N-diethylcarbamoyl)glutathione (carbamathione).

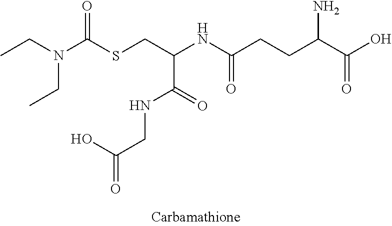 [SMILES: CCN(CC)C(=O)SCC(NC(=O)CCC(N)C(=O)O)C(=O)NCC(=O)O]

In some embodiments, the salt form of S—(N,N-diethylcarbamoyl)glutathione is an acid addition salt form or a base addition salt form.

In some embodiments, the salt form of S—(N, N-diethylcarbamoyl)glutathione is defined to include all forms of the compound including, but not limited to, hydrates, solvates, isomers (including for example rotational stereoisomers), crystalline, co-crystalline, semi-crystalline, and non-crystalline, amorphous forms, isomorphs, eutectics, polymorphs, metabolites and prodrugs thereof. For example, it may exist in unsolvated and solvated forms with pharmaceutically acceptable solvents such as water, ethanol and the like. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm. In general, the solvated forms are considered equivalent to the unsolvated forms for the purposes of the present invention. In preferred embodiments, the salt form of S—(N, N-diethylcarbamoyl)glutathione is crystalline, co-crystalline, semi-crystalline or an amorphous powder.

In some embodiments, the salt form of S—(N,N-diethylcarbamoyl)glutathione (carbamathione) is prepared by treating the neutral form with an appropriate acid, such as an inorganic acid or an organic acid. Inorganic acids include, but are not limited to hydrochloric acid, hydrobromic acid, sulfuric acid, nitric acid, phosphoric acid, thiocyanic acid and the like. Organic acids include, but are not limited to, 2,2-dichloroacetic acid, ascorbic acid, aspartic acid, acetic acid, adipic acid, benzenesulfonic acid, benzoic acid, 4-acetamido-benzoic acid, camphoric acid, camphor-10-sulfonic acid, capric acid (decanoic acid), caproic acid (hexanoic acid), caprylic acid (octanoic acid), carbonic acid, cinnamic acid, cyclamic acid, citric acid, ethane-1,2-disulfonic acid, ethanesulfonic acid, ethanedisulfonic acid, 2-hydroxy-ethanesulfonic acid, naphthalenesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, naphthalene-1,5-disulfonic acid, naphthalene-1-sulfonic acid, naphthalene-2-sulfonic acid, 1-hydroxy-2-naphthoic acid, nicotinic acid, oleic acid, orotic acid, oxalic acid, palmitic acid, pamoic acid (embonic acid), propionic acid, pyroglutamic acid, salicyclic acid, 4-aminosalicyclic acid, sebacic acid, sorbic acid, succinic acid, stearic acid, tartaric acid, toluenesulfonic acid monohydrate and undecylenic acid, or its solvates, polymorphs, hydrates or mixtures thereof.

In another aspect, the disclosure relates to a base addition salt of S—(N,N-diethylcarbamoyl)glutathione (carbamathione). In some embodiments, base addition salt forms of S—(N,N-diethylcarbamoyl)glutathione (carbamathione) are prepared by treatment of the neutral compound with organic or inorganic bases. Inorganic bases include by way of example only, sodium, potassium, lithium, ammonium, calcium and magnesium salts. Organic bases include, but are not limited to, primary, secondary and tertiary amines, such as alkyl amines, dialkyl amines, trialkyl amines, substituted alkyl amines, di(substituted alkyl) amines, and tri(substituted alkyl) amines. Organic bases also include quaternary ammonium bases such as choline salts (e.g., 2-hydroxyethyl)trimethylammonium hydroxide). In certain such embodiments, also included are amines where the two or three substituents, together with the amino nitrogen, form a heterocyclic group. In certain such embodiments, suitable amines include, by way of example only, isopropylamine, trimethyl amine, diethyl amine, tri(iso-propyl) amine, tri(n-propyl) amine, ethanolamine, 2-dimethyl aminoethanol, deanol (dimethylethanolamine), tromethamine, L-lysine, L-arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. In preferred embodiments, the base addition salt of S—(N,N-diethylcarbamoyl)glutathione is a L-lysine salt.

In some embodiments, the salt is selected from the group consisting of an acetate salt, an adipate salt, an ascorbate salt, a benzoate salt, a camphorate salt, a citrate salt, a fumarate salt, a glutarate salt, a glycolate salt, a hydrochloride salt, a tartrate salt, a malate salt, a maleate salt, a methanesulfonate salt, an ethanedisulfonate salt, an ethanesulfonate salt, a naphthalenesulfonate salt, an oxalate salt, a phosphate salt, a sulfate salt, a sorbate salt, a benzenesulfonate, a cyclamate salt, succinate salt, a toluenesulfonate salt, an arginine salt, a lysine salt, a deanol salt, a choline salt, a sodium salt, a potassium salt, a diethylammonium salt, a meglumine salt, a pyridoxine salt, and a tris(hydroxymethyl)ammonium salt its solvates, polymorphs, hydrates or mixtures thereof. In a preferred embodiment, the salt is an L-lysine salt or a solvate, polymorph, hydrate or mixture thereof. In some embodiments, the L-lysine salt is a hydrate.

In some embodiments, the salt form of S—(N, N-diethylcarbamoyl)glutathione has increased solubility as compared to free S—(N, N-diethylcarbamoyl)glutathione. In some embodiments the solubility of the salt form is between about 5% and 100% higher than the solubility of free S—(N, N-diethylcarbamoyl)glutathione, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100% higher than free S—(N, N-diethylcarbamoyl)glutathione. A given percent increase in the solubility of the salt form of S—(N, N-diethylcarbamoyl)glutathione means the amount of solute that can be dissolved in solution increases by that percent (i.e., 5%, 10%, 15%, 20%, 25%, 30%, 35%, 40%, 45%, 50%, 55%, 60%, 65%, 70%, 75%, 80%, 85%, 90%, 95% or 100%) as compared to free S—(N, N-diethylcarbamoyl)glutathione in a solution having the same properties (e.g., solvent, temperature, pH, etc). For example, if 10 mg of S—(N, N-diethylcarbamoyl)glutathione dissolve in 1 mL of 25° C. water having a pH of 7.0, and 15 mg of S—(N, N-diethylcarbamoyl)glutathione dissolve in 1 mL of 25° C. water having a pH of 7.0 when added as a salt form, then the solubility of S—(N, N-diethylcarbamoyl)glutathione has increased by 50%.

### Pharmaceutical Composition of the Disclosure

In one aspect, the invention relates to a pharmaceutical composition comprising a therapeutically effective amount of a salt form of S—(N,N-diethylcarbamoyl)glutathione and at least one pharmaceutically acceptable carrier. In some embodiments, the salt is selected from the group consisting of an acetate salt, an adipate salt, an ascorbate salt, a benzoate salt, a camphorate salt, a citrate salt, a fumarate salt, a glutarate salt, a glycolate salt, a hydrochloride salt, a tartrate salt, a malate salt, a maleate salt, a methanesulfonate salt, an ethanedisulfonate salt, an ethanesulfonate salt, a naphthalenesulfonate salt, an oxalate salt, a phosphate salt, a sulfate salt, a sorbate salt, a benzenesulfonate, a cyclamate salt, succinate salt, a toluenesulfonate salt, an arginine salt, a lysine salt, a deanol salt, a choline salt, a sodium salt, a potassium salt, a diethylammonium salt, a meglumine salt, a pyridoxine salt, and a tris(hydroxymethyl)ammonium salt its solvates, polymorphs, hydrates or mixtures thereof. In a preferred embodiment, the salt is an L-lysine salt or a solvate, polymorph, hydrate or mixture thereof. In some embodiments, the L-lysine salt is a hydrate.

Examples of acceptable carriers include, but are not limited to, a solid, gelled or liquid diluent or an ingestible capsule. Suitable excipients include, but are not limited to, starch, glucose, lactose, sucrose, mannitol, sorbitol, gelatin, malt, rice, flour, chalk, silica gel, sodium stearate, glycerol monostearate, talc, sodium chloride, dried skim milk, glycerol, propylene, glycol, water, polyvinyl alcohol, polyethylene glycol, omega 3-oils, ethanol and the like.

Alternatively, compositions described herein may be formulated as a lyophilizate, or compounds may be encapsulated within liposomes using technology known in the art. Pharmaceutical compositions may also contain other components, which may be biologically active or inactive. Such components include, but are not limited to, buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, stabilizers, dyes, flavoring agents, and suspending agents, agents that form eutectics and/or preservatives.

A eutectic is a mixture of chemical compounds or elements that has a single chemical composition that melts at a lower temperature than any other composition made up of the same ingredients. A composition comprising a eutectic is known as the eutectic composition and its melting temperature is known as the eutectic temperature. In some embodiments, the salt form of S—(N, N-diethylcarbamoyl)glutathione is part of a eutectic composition.

The pharmaceutical compositions of the invention may be prepared in many forms that include, but are not limited to, tablets, such as scored tablets, coated tablets, or orally dissolving tablets; thin films, caplets, capsules (e.g. hard or soft gelatin capsules), troches, dragees, dispersions, suspensions, aqueous solutions, liposomes, patches and the like, including sustained release formulations well known in the art.

Oral liquid pharmaceutical compositions may be in the form of, for example, aqueous or oily suspensions, solutions, emulsions, syrups or elixirs, or may be presented as a dry product for constitution with water or other suitable vehicle before use.

In some embodiments, when the pharmaceutical composition comprising a therapeutically effective amount of a salt form of S—(N,N-diethylcarbamoyl)glutathione is orally administered, the pharmaceutical composition is safe, stable and bioavailable. Bioavailability refers to the fraction of an administered dose of unchanged drug that reaches the systemic circulation. In some embodiments, the pharmaceutical composition comprising a therapeutically effective amount of a salt form of S—(N,N-diethylcarbamoyl)glutathione is at least 80% absorbed in about 1 hour following the administration. In another embodiment, the pharmaceutical composition comprising a therapeutically effective amount of a salt form of S—(N,N-diethylcarbamoyl)glutathione is at least 80% absorbed in about 2 hours following the administration. In another embodiment, the pharmaceutical composition comprising a therapeutically effective amount of a salt form of S—(N,N-diethylcarbamoyl)glutathione is at least 80% absorbed in about 3 hours following the administration.

The compounds according to the invention may also be formulated for parenteral administration. Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, etc. Parenteral formulations may be presented in unit dosage form in ampules, prefilled syringes, small volume infusion containers or multi-dose containers with an added preservative.

Any suitable excipient or carrier for subcutaneous administration known to those of ordinary skill in the art for use in pharmaceutical compositions may be employed in the compositions described herein.

For topical administration to the epidermis, the compounds may be formulated as ointments, creams or lotions, or as the active ingredient of a transdermal patch. Suitable transdermal delivery systems are disclosed, for example, in A. Fisher et al. (U.S. Pat. No. 4,788,603). Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Pharmaceutical compositions suitable for topical administration in the mouth include unit dosage forms such as lozenges comprising the compound of the invention in a flavored base, usually sucrose and acadia or tragacanth; pastilles comprising the compound in an inert base such as gelatin and glycerin or sucrose and acacia; mucoadherent gels, and mouthwashes comprising the compound in a suitable liquid carrier.

In some embodiments, the above-described pharmaceutical compositions can be formulated for sustained or slow release of the compound. Sustained-release formulations may contain an agent dispersed in a carrier matrix and/or contained within a reservoir surrounded by a rate controlling membrane. Excipients for use within such formulations are biocompatible and may also be biodegradable; preferably the formulation provides a relatively constant level of active component release. The amount of active compound contained within a sustained release formulation depends upon the site of implantation, the rate and expected duration of release, and the nature of the condition to be treated or prevented.

Pharmaceutical compositions suitable for rectal administration wherein the carrier is a solid are most preferably presented as unit dose suppositories. Suitable carriers include cocoa butter and other materials commonly used in the art. The suppositories may be conveniently formed by admixture of the active compound with the softened or melted carrier(s) followed by chilling and shaping in molds.

For administration by inhalation, the compounds according to the present disclosure are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

For intranasal administration, the compounds of the invention may be administered as a liquid spray or as an oil spray (e.g., castor oil), such as via a plastic bottle atomizer.

Pharmaceutical compositions of the invention may also contain conventional adjuvants such as suspending agents, emulsifying agents, non-aqueous vehicles (which may include edible oils), flavorings, colorings, antimicrobial agents, or preservatives.

### Method of Producing a Salt Form of S—(N,N-diethylcarbamoyl)glutathione

A salt form of S—(N, N-diethylcarbamoyl)glutathione can be produced by methods known to those skilled in the art. For example, dissolving S—(N,N-diethylcarbamoyl)glutathione in a suitable solvent, followed by the addition of stoichiometric equivalents or an excess of an acid or a base can result in the formation of a salt form of S—(N,N-diethylcarbamoyl)glutathione by virtue of the carboxylic acid groups, thiol group and/or amino groups. The addition of the acid or the base can be to a solution, a suspension, or a slurry comprising S—(N,N-diethylcarbamoyl)glutathione. Further, the salt form can be isolated according to any number of methods known to those skilled in the art. For example, an anti-solvent can be added to the mixture to induce precipitation of the salt form, which can subsequently be filtered. The precipitate can be crystalline, semi-crystalline or amorphous. Alternatively, crystallization techniques such as, but not limited to liquid-liquid diffusion, vapor-liquid diffusion, and slow evaporation can result in the formation of a crystalline salt, which can then be isolated via filtration or removal of the supernate. Grinding and kneading experiments can also result in the formation of a salt form. For example, S—(N,N-diethylcarbamoyl)glutathione can be ground with a catalytic amount of a suitable solvent by ball milling in the presence of one equivalent, or an excess, of the selected acid or base co-former. Analyzing the recovered solids by XRPD will allow for the determination of new salt forms of S—(N,N-diethylcarbamoyl)glutathione.

In one aspect, the invention relates to a method of producing an acid addition salt of S—(N, N-diethylcarbamoyl)glutathione, comprising:
(i) suspending S—(N, N-diethylcarbamoyl)glutathione in a C1-C6 alcohol, dichloromethane, water or an aqueous lower alcohol, to thereby form a suspension;
(ii) adding to the suspension an acid, to thereby form a mixture; and
(iii) optionally adding tert-butyl methyl ether, cyclohexane, acetonitrile, acetone, or an acetonitrile-acetone mixed solvent to the mixture, to thereby crystallize the salt, or lyophilizing the mixture.

In some embodiments, the disclosure relates to a method of producing a salt form of S—(N,N-diethylcarbamoyl)glutathione, comprising mixing S—(N,N-diethylcarbamoyl)glutathione and an appropriate amount of acid in the presence of a suitable solvent. In some embodiments, the method comprises grinding or kneading S—(N,N-diethylcarbamoyl)glutathione with one equivalent of acid. In some embodiments, the method comprises grinding or kneading S—(N,N-diethylcarbamoyl)glutathione with an excess of acid. In some embodiments, the solvent is water, ethanol, methanol or dichloromethane. In some embodiments, the method further comprises evaporating the solvent from the mixture.

In some embodiments, the acid is selected from the group consisting of hydrobromic acid, nitric acid, 2,2-dichloroacetic acid, ascorbic acid, aspartic acid, acetic acid, adipic acid, benzenesulfonic acid, benzoic acid, 4-acetamido-benzoic acid, camphoric acid, camphor-10-sulfonic acid, capric acid (decanoic acid), caproic acid (hexanoic acid), caprylic acid (octanoic acid), carbonic acid, cinnamic acid, cyclamic acid, citric acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, hydrochloric acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid (embonic acid), phosphoric acid, propionic acid, pyroglutamic acid, salicyclic acid, 4-aminosalicyclic acid, sebacic acid, sorbic acid, succinic acid, stearic acid, sulfuric acid, tartaric acid, thiocyanic acid, toluenesulfonic acid monohydrate, undecylenic acid, N-cyclohexylsulfamic acid, camphor-10-sulfonic acid, naphthalenedisulfonic acid, and quinaldic acid, or its solvates, polymorphs, hydrates or mixtures thereof.

In some embodiments, the acid is selected from the group consisting of acetic acid, adipic acid, ascorbic acid, benzoic acid, camphoric acid, citric acid, fumaric acid, glutaric acid, glycolic acid, hydrochloric acid, tartaric acid, malic acid, maleic acid, methanesulfonic acid, oxalic acid, phosphoric acid, sulfuric acid, sorbic acid, succinic acid, toluenesulfonic acid monohydrate, N-cyclohexylsulfamic acid, camphor-10-sulfonic acid, naphthalenedisulfonic acid, and quinaldic acid or its solvates, polymorphs, hydrates or mixtures thereof.

In one aspect, the invention relates to a method of producing a salt form of S—(N,N-diethylcarbamoyl)glutathione, comprising:
(i) suspending S—(N, N-diethylcarbamoyl)glutathione in a C1-C6 alcohol, water, dichloromethane or an aqueous lower alcohol, to thereby form a suspension;
(ii) adding to the suspension a base, to thereby form a mixture; and
(iii) optionally adding tert-butyl methyl ether, cyclohexane, acetonitrile, acetone, or an acetonitrile-acetone mixed solvent to the mixture, to thereby crystallize the salt, or lyophilizing the mixture.

In some embodiments, the base is an inorganic base is selected from sodium, potassium, lithium, ammonium, calcium and magnesium salts, isopropylamine, trimethylamine, diethylamine, tri(iso-propyl) amine, tri(n-propyl) amine, ethanolamine, 2-dimethyl aminoethanol, deanol (dimethylethanolamine), tromethamine, L-lysine, L-arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

In some embodiments the base is selected from the group consisting of sodium hydroxide, potassium hydroxide, choline hydroxide, L-arginine, L-lysine, deanol, diethylamine and tromethamine. In some embodiments, the base is L-lysine.

In some embodiments, the disclosure relates to a method of producing a salt form of S—(N,N-diethylcarbamoyl)glutathione, comprising mixing S—(N,N-diethylcarbamoyl)glutathione and an appropriate amount of base in the presence of a suitable solvent. In some embodiments, the method comprises grinding or kneading S—(N,N-diethylcarbamoyl)glutathione with one equivalent of base. In some embodiments, the method comprises grinding or kneading S—(N,N-diethylcarbamoyl)glutathione with an excess of base. In some embodiments, the solvent is water, ethanol, methanol or dichloromethane. In some embodiments, the method further comprises evaporating the solvent from the mixture. In some embodiments, the base is L-lysine.

### Prevention or Treatment of a Glutamate-Related Disorder

In one aspect, the disclosure relates to a method of preventing or treating a glutamate-related disorder in a subject in need thereof or at risk thereof, comprising administering to said subject a therapeutically effective amount of a salt form of S—(N,N-diethylcarbamoyl)glutathione.

In some embodiments, the subject in need of treatment or at risk of having the disorder include, but is not limited to, mammals, such as humans, primates, livestock animals (including bovines, porcines, camels, etc.), companion animals (e.g., canines, felines, etc.) and rodents (e.g., mice and rats). In one embodiment, the compound is administered to a mammal, preferably a human.

In some embodiments, the pharmaceutical composition of the invention may be administered by standard routes of administration. Many methods may be used to introduce the formulations into a subject, these include, but are not limited to, intranasal, intratracheal, sublingual, oral, intradermal, intrathecal, intramuscular, transdermal, rectal, intraperitoneal, intravenous, conjunctival and subcutaneous routes.

It will be further appreciated that the amount of the present compound(s), a combination of the present compounds, or the active salt or derivative thereof, required for use in the prevention or treatment will vary not only with the particular salt selected but also with the route of administration, the nature of the condition being treated and the age and condition of the patient and will be ultimately at the discretion of the attendant physician or clinician.

The amount of the composition of the invention or a combination thereof that is administered and the frequency of administration to a given subject will depend upon a variety of variables related to the patient's psychological profile and physical condition. For evaluations of these factors, see Brien, J F et al., Eur J Clin Pharmacol. 1978; 14(2):133-41; and Physicians' Desk Reference, Charles E. Baker, Jr., Pub., Medical Economics Co., Oradell, N.J. (41st ed., 1987).

The dose of the composition for preventing or treating a glutamate related disease may be determined according to parameters understood by a skilled person in the medical art.

In some embodiments, the invention provides a method wherein the salt form of S—(N,N-diethylcarbamoyl)glutathione is present in the pharmaceutical composition in an amount from 0.5 mg to 500 mg/kg. In certain embodiments, the salt form of S—(N,N-diethylcarbamoyl)glutathione is present in the composition in an amount from 0.5 to 50 mg/kg. In certain embodiments, the salt form of S—(N,N-diethylcarbamoyl)glutathione is present in the composition in an amount from 0.5 to 20 mg/kg. In certain embodiments, the salt form of S—(N,N-diethylcarbamoyl) glutathione is present in the composition in an amount from 5 to 100 mg/kg. In some embodiments, the salt form of S—(N,N-diethylcarbamoyl) glutathione is present in the composition in an amount from 10 to 800 mg/kg. In other embodiments, the salt form of S—(N,N-diethylcarbamoyl) glutathione is present in the composition in an amount from 50 to 800 mg/kg. In some embodiments, the salt form of S—(N,N-diethylcarbamoyl) glutathione is present in the composition in an amount from 50 to 250 mg/kg. In other embodiments, the salt form of S—(N,N-diethylcarbamoyl)glutathione is present in the composition in an amount from 200 to 700 mg/kg. In another embodiment, the amount is from 400 to 700 mg/kg. In some embodiments, the amount is from 500 to 700 mg/kg. In some embodiments, the amount is from 600 to 700 mg/kg.

In some embodiments, the peak plasma level of the salt form of S—(N,N-diethylcarbamoyl)glutathione, after administration, is in the range of 2 to 100 nmol/L. In another embodiment, the range is from 5 to 50 nmol/L. In another embodiment, the range is from 5 to 100 nmol/L. In another embodiment, the range is from 1 to 10 μmol/L. In other embodiments, the range is from 10 to 1000 μmol/L. In certain embodiments, the range is from 50 to 800 μmol/L. In some embodiments, the range is from 200 to 700 μmol/L. In another embodiment, the range is from 200 to 500 μmol/L. In other embodiments, the range is from 400 to 700 μmol/L. In some embodiments, the range is from 500 to 700 μmol/L. In some embodiments, the range is from 600 to 700 μmol/L.

In some embodiments, the average area under the curve (AUC) after the administration of the salt form of S—(N, N-diethylcarbamoyl) glutathione is between 20 and 1000 h μg/ml. In other embodiments, the AUC is between 30 and 800 h μg/ml. In other embodiments, the AUC is between 50 and 700 h μg/ml. In other embodiments, the AUC is between 70 and 500 h μg/ml. In other embodiments, the AUC is between 80 and 400 h μg/ml. In other embodiments, the AUC is between 100 and 300 h μg/ml.

In some embodiments, the trough plasma level of the salt form of S—(N,N-diethylcarbamoyl)glutathione after administration is in the range of 2 to 100 nmol/L. In another embodiment, the range is from 5 to 50 nmol/L. In another embodiment, the range is from 5 to 100 nmol/L. In another embodiment, the range is from 1 to 10 μmol/L. In other embodiments, the range is from 10 to 1000 μmol/L. In certain embodiments, the range is from 50 to 800 μmol/L. In some embodiments, the range is from 200 to 700 μmol/L. In another embodiment, the range is from 200 to 500 μmol/L. In other embodiments, the range is from 400 to 700 μmol/L. In some embodiments, the range is from 500 to 700 μmol/L. In some embodiments, the range is from 600 to 700 μmol/L.

In some embodiments, examples of glutamate related disorders include, but are not limited to Huntington's disease, Alzheimer's disease, Parkinson's disease, acquired immunodeficiency syndrome (AIDS) neuropathy, epilepsy, an eating disorder, a sleep disorder, nicotine addiction, cerebral ischemia (stroke), familial Amyotrophic Lateral Sclerosis (ALS), Wemicke-Korsakoff syndrome, cerebral beriberi, Machado-Joseph disease, Soshin disease, anxiety, glutamate related convulsions, hepatic encephalopathy, neuropathic pain, domoic acid poisoning, hypoxia, anoxia, mechanical trauma to the nervous system, hypertension, alcohol withdrawal seizures, alcohol addiction, alcohol craving, cardiovascular ischemia, oxygen convulsions, hypoglycemia, Creutzfeldt-Jakob disease, cocaine addiction, noise induced hearing loss, heroin addiction, addiction to opioids, cyanide-induced apoptosis, schizophrenia, bipolar disorder, peripheral neuropathy associated with diabetes and non-ketonic hyperglycinemia.

In some embodiments, the glutamate-related disorder is selected from the group consisting of anxiety, glutamate related convulsions, hepatic encephalopathy, domoic acid poisoning, hypoxia, anoxia, alcohol addiction, alcohol withdrawal seizures, alcohol craving, oxygen-induced seizures and hypoglycemia.

In some embodiments, the glutamate-related disorder is an alcohol use disorder (AUD). In some embodiments, the alcohol use disorder is selected from the group of alcohol addiction, alcohol abuse, alcohol dependence, alcohol withdrawal seizures and alcohol craving.

AUD can result in symptoms including dyspepsia or epigastric pain, headache, diarrhea, difficulty in sleeping, fatigue, unexplained weight loss, apparent malnutrition, easy bruising, increased mean corpuscular volume, elevated transaminase levels (especially an aspartate transaminase level greater than of alanine transaminase), elevated y-glutamyl transferase levels, iron-deficiency anemia, hepatomegaly, jaundice, spider angiomata, ascites, and peripheral edema. Behavioral symptoms associated with AUD include absenteeism from work or school increasing irritability, difficulties with relationships, verbal or physical abuse and depression.

To be diagnosed with AUD, individuals must meet certain criteria outlined in the Diagnostic and Statistical Manual of Mental Disorders (DSM). Under DSM-5 (current manual), anyone meeting any two of the 11 criteria during the same 12-month period receives a diagnosis of AUD. The severity of AUD (mild, moderate, or severe) is based on the number of criteria met. See Table below.

| Criteria | In the past year, have you: |
|---|---|
| 1 | Had times when you ended up |
| | drinking more, or longer than you |
| | intended? |
| 2 | More than once wanted to cut |
| | down or stop drinking, or tried to, but |
| | couldn't? |
| 3 | Spent a lot of time drinking? |
| | Or being sick or getting over the |
| | aftereffects? |
| 4 | Experienced craving—a strong |
| | need, or urge, to drink? |
| 5 | Found that drinking—or being sick |
| | from drinking—often interfered |
| | with taking care of your home or |
| | family? Or caused job troubles? Or |
| | school problems? |
| 6 | Continued to drink even though it |
| | was causing trouble with your |
| | family or friends? |
| 7 | Given up or cut back on activities |
| | that were important or interesting |
| | to you, or gave you pleasure, in |
| | order to drink? |
| 8 | More than once gotten into situations |
| | while or after drinking that |
| | increased your chances of getting |
| | hurt (such as driving, swimming, |
| | using machinery, walking in a |
| | dangerous area, or having unsafe sex)? |
| 9 | Continued to drink even though it |
| | was making you feel depressed or |
| | anxious or adding to another health |
| | problem? Or after having had a |
| | memory blackout? |
| 10 | Had to drink much more than you |
| | once did to get the effect you want? |
| | Or found that your usual number |
| | of drinks had much less effect than |
| | before? |
| 11 | Found that when the effects of |
| | alcohol were wearing off, you had |
| | withdrawal symptoms, such as |
| | trouble sleeping, shakiness, |
| | irritability, anxiety, depression, |
| | restlessness, nausea, or sweating? Or |
| | sensed things that were not there? |

Currently disulfram (DSF) is commonly used in the treatment of AUD. The efficacy of DSF in the treatment of AUD has been attributed to its inhibitory activity on aldehyde dehydrogenase (ALDH₂). However, this is also the mechanistic basis of most safety concerns regarding DSF. Specifically, DSF's inhibition of hepatic mitochondrial ALDH₂ blocks the second step in alcohol metabolism. Thus, any subsequent consumption of ethanol results in an accumulation of the toxic intermediate, acetaldehyde. This produces the adverse effect known as the disulfiram-ethanol reaction (DER) when ethanol is consumed by patients being treated with DSF. Specifically, acetaldehyde accumulation results in a potent systemic vasodilatory response with symptoms such as flushing, headache, nausea, and tachycardia (US 2013/0165511 A1). Carbamathione, by contrast, is devoid of inhibitory activity of ALDH₂ (Faiman et al., 2013. Neuropharmacology 75; 95-105), and therefore has no risk for DERs.

Once ingested, DSF is metabolized into S-methyl-N,N-diethiolcarbamate sulfoxide (DETC-MeSO), which is further metabolized into carbamathione (Jin et al., 1994; Nagendra et al., Biochem. Pharmacol. 55: 749-756, 1998). In microdialysis studies in rats, intravenous carbamathione administration increases dopamine (DA), decreases GABA and has a biphasic effect on glutamate (Glu) in the nucleus accumbens (NAc) and prefrontal cortex (PFC), two brain regions implicated in the rewarding process associated with AUDs (Faiman et al., Neuropharmacology. 75: 95-105, 2013). Administration of prodrug DSF also produces these same changes in DA, GABA, and GLu in the NAc and PFC. When DSF metabolism is inhibited, carbamathione is not formed, and no changes in these neurotransmitters occur (Faiman et al., 2013. Neuropharmacology 75; 95-105). Without wishing to be bound by theory, the efficacy of DSF in the treatment of AUD may be due to the downstream formation of the carbamathione metabolite after DSF is administered to a patient, and its subsequent effect on DA, GABA, and GLU and/or other neurotransmitters. Accordingly, in one aspect of this disclosure, the administration of carbamathione, or a pharmaceutically acceptable salt thereof, instead of DSF is also effective in the treatment of AUD while concurrently avoiding the adverse side effects associated with ALDH₂ inhibition and the DERs associated therewith.

In some embodiments, the composition comprising a salt form of S—(N,N-diethylcarbamoyl)glutathione is administered at least 30 minutes in advance of the usual drinking time. In some embodiments, the composition comprising a salt form of S—(N,N-diethylcarbamoyl)glutathione is administered at least 2 hours in advance of the usual drinking time.

In some embodiments, any of the prevention or treatment methods described may be combined with psychotherapeutic intervention to improve the outcome of the prevention or of the treatment.

In one embodiment, the compound is administered in combination with one or more therapeutic agents useful in the prevention or treatment of a glutamate related disorder.

The expression “in combination”, as used herein, is to be understood that the compound of the invention can be administered together or separately, simultaneously, concurrently or sequentially with a therapeutic agent useful in the prevention or treatment of a glutamate related disease.

A person skilled in the art understands that the combined administration of the compound of the invention and an additional therapeutic agent useful in the prevention or treatment of a glutamate related disorder can be in the form of a single dosage form or in separate dosage forms.

Examples of therapeutic agents that can be administered in combination with the salt form of S—(N,N-diethylcarbamoyl)glutathione include, but are not limited to gabapentin and topiramate, acamprosate, coprine, cyanamide, cyclobenzaprine, naltrexone, rasagiline and selegiline or pharmaceutically acceptable salts thereof.

### EXAMPLES

### Example 1. Efficacy of Carbamathione in Reducing Ethanol Intake of Mice or Rats

The efficacy of carbamathione in reducing ethanol intake of rats was assessed. Adult male alcohol-preferring rats (P rats) and high alcohol-drinking-1 (HAD1) rats (˜75 days of age at the start) were used in this study. These rats underwent an 8-week acquisition/acclimation period of concurrent free-choice access to 15% and 30% ethanol. Animals were initiated with 24-hour access, which was titrated down to 2 hours per day for 5 days (Monday-Friday)/week access. Ethanol access began at the beginning of the dark cycle (10:00 h) in a room maintained on a reverse dark-light cycle (10:00 h to 22:00 h lights out).

After the acquisition period, the animals underwent three weeks of testing. Four doses were tested: 0, 100, 200, and 400 mg/kg/day. Sterile isotonic (0.9% normal) saline with 0.25% T_{ween}® 80 was used as the vehicle for all doses. Injection solutions were prepared approximately 1 hour prior to each administration.

The carbamathione solution was maintained at −20° C. until mixing the solutions each day. Adding the carboxyl-associated compound helped dissolve the carbamathione solid, which was pulverized by mortar and pestle with 50 μl Tween® 80, resulting in a pH of 3.5. Neutralizing to a pH 7.0 on a stir plate allowed for the compound to stay in solution. Doses were calculated at 3 ml/kg to allow for injection volume of 1.5 ml per 500 g rat. The dose groups were balanced for ethanol intake using data from the last week of acquisition. The drug was administered intraperitoneally (IP) once daily (Monday through Friday) 30 min prior to lights out. Food and water were available ad libitum.

Data were analyzed by Dose, by Test Day, and 2-way mixed ANOVAs on each rat line, followed by Dunnett t-test planned comparisons.

For P rats, other than the significant main effect of dose (p=0.021), which determined close to the 25% of the variance (effect-size=0.235, with a Power of 0.757) in the 2-way ANOVA, there were no significant repeated measure effects of carbamathione. See Table 1. For the main effect of dose, Dunnett's t-test revealed a significant effect for the highest dose.

TABLE 1
IP carbamathione P Rats: 2 h Ethanol Intake

| | F(df) | F-statistic | p-value | *Effect Size | Power |
|---|---|---|---|---|---|
| Dose × | F(12, 144) | 1.357 | NS, p = 0.193 | 0.102 | 0.730 |
| Test Day | | | | | |
| Test Day | F(4, 144) | 1.457 | NS, p = 0.219 | 0.039 | 0.444 |
| Dose | F(3, 36) | 3.680 | p = 0.021 | 0.235 | 0.757 |

*Effect size: Partial eta-squared for MANOVA;
NS: not-significant

For HAD-1 rats, other than the significant main effect of Test Day (p=0.036), which determined close to the 7% of the variance (effect-size=0.068, with a Power of 0.727) in the 2-way ANOVA, there were no significant repeated measure effects of carbamathione. See Table 2.

TABLE 2
IP carbamathione HAD-1 Rats: 2 h Ethanol Intake

| | F(df) | F-statistic | p-value | *Effect Size | Power |
|---|---|---|---|---|---|
| Dose × | F(12, 144) | 0.695 | NS, p = 0.754 | 0.102 | 0.730 |
| Test Day | | | | | |
| Test Day | F(4, 144) | 2.644 | p = 0.036 | 0.039 | 0.444 |
| Dose | F(3, 36) | 0.495 | p = 0.688 | 0.040 | 0.140 |

*Effect size: Partial eta-squared for MANOVA;
NS: not-significant

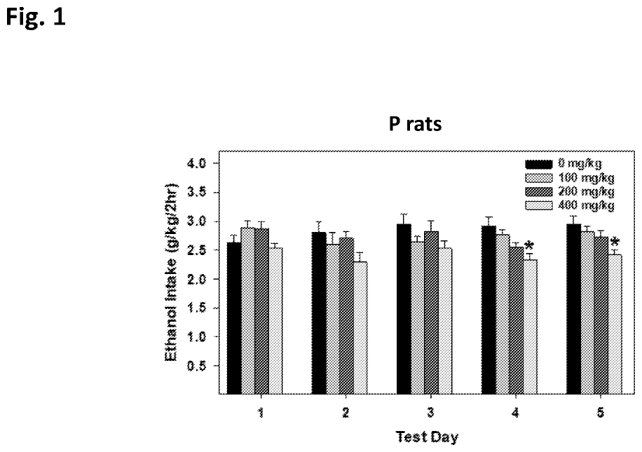

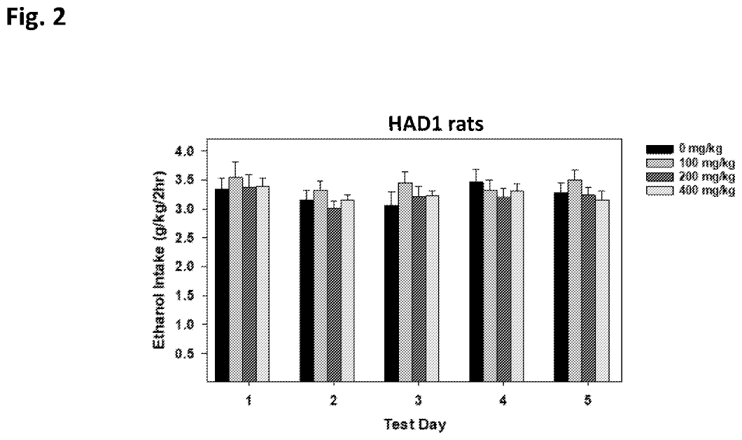

As can be seen in FIGS. 1 and 2, free carbamathione has a modest positive effect in P rats, but has no effect in HAD1 rats. This modest effect is attributable to the limited solubility of carbamathione in solution. The addition of Tween® 80 resulted in the formation of a foamy suspension that may result in under-dosing or poor absorption of carbamathione.

In order to determine if the absorption of carbamathione is responsible for the modest results observed in the previous study, a different vehicle (0.25% carboxymethyl cellulose (CMC) in water) was tested.

Following the established protocol, adult male C57BL/6J mice (N=96) were trained to drink ethanol in a limited access (2 hour/day) free-choice (15% v/v ethanol vs. water) drinking procedure. After four weeks, stable baseline level of intake was established, and mice were separated into two groups. One group of mice (CIE group) was exposed to chronic intermittent ethanol (CIE) vapor exposure in inhalation chambers (16 hour/days x 4 days). The remaining mice (CTL group) were treated similarly but exposed to air in inhalation chambers. After a 72 hour forced abstinence period, all mice resumed ethanol drinking in the same limited access paradigm for a 5-day test period. This pattern of weekly CIE (or air) exposure cycles with intervening weekly test drinking cycles was repeated for seven cycles following procedures previously published (Becker and Lopez, 2004; Griffin et al., 2009; Lopez and Becker, 2005).

All mice received intraperitoneal (IP) administration of saline 30 min prior to the start of daily drinking sessions during baseline and the early test cycles to acclimate the animals to the handling procedure. After the fourth ethanol intake test cycle, mice were further divided into carbamathione dose treatment conditions (N=10-12/group).

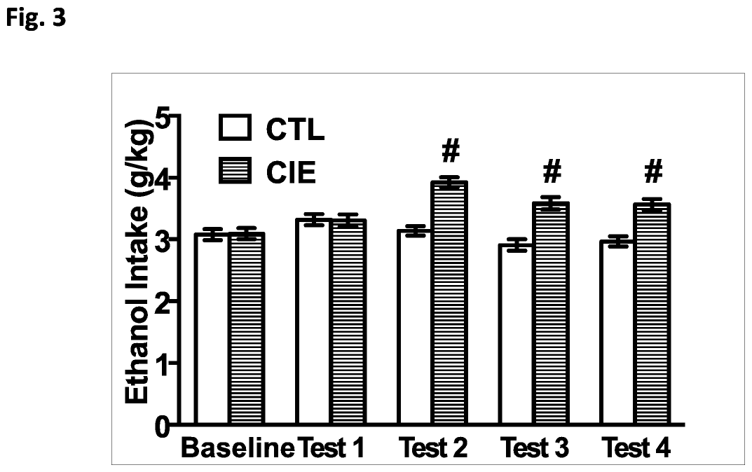

Weekly average ethanol intake (g/kg) during the last week of baseline and early test cycles was analyzed by analysis of variance (ANOVA), with Group (CTL, CIE) as a between-subjects factor and Phase (Baseline—Test 4) as a repeated measure. ANOVA indicated significant main effects of Group [F(1,84)=18.88; p<0.0001], Phase [F(4,336)=10.88; p<0.0001] and a significant interaction between these factors [F(4,336)=15.48; p<0.0001]. Newman-Keuls post-hoc comparisons indicated that there was no difference in ethanol intake between groups during baseline—an expected outcome since mice were separated into CIE and CTL groups based on their baseline level of intake. CTL mice showed a stable level of intake throughout the study. In contrast, CIE mice consumed significantly more ethanol during Test cycles 2, 3, and 4 compared to their own baseline and compared to CTL mice during the same test cycle (# in FIG. 3).

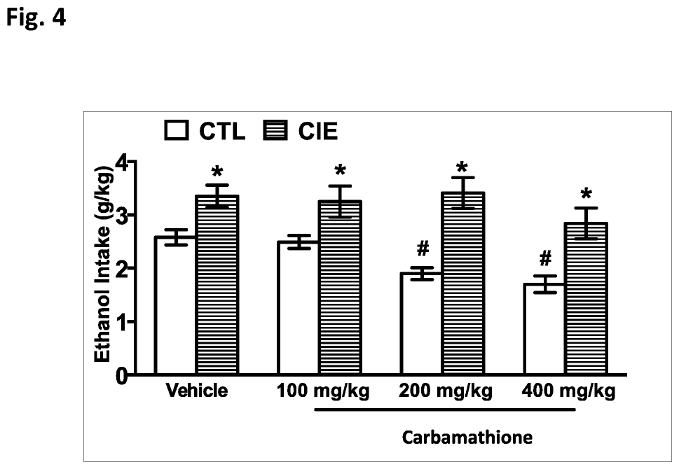

After Test 4, CIE and CTL mice were separated in dose groups for Test 5 (N=11-12/group), with the groups equated for intake during Test 4. Mice received intraperitoneal (IP) injections of carbamathione (100, 200 or 400 mg/kg) or vehicle (0.25% carboxymethyl cellulose, CMC in water) 30 min before drinking. The carbamathione IP injections were administered as a suspension. Ethanol intake during Test 5 was averaged for the week and analyzed by ANOVA, with Group (CTL, CIE) and carbamathione dose (0, 100, 200, 400 mg/kg) as between-subject factors. ANOVA indicated a significant main effect of Group [F(1,78)=53.33; p<0.0001], reflecting a higher level of ethanol intake in CIE mice compared to CTL mice (* in FIG. 4). ANOVA also indicated a significant effect of carbamathione dose [F(3,78)=4.39; p<0.01]. Post hoc tests indicated significantly lower ethanol intake in mice that received the highest dose of carbamathione (400 mg/kg) compared to mice that received vehicle and the lowest dose of carbamathione (100 mg/kg). While the group by carbamathione dose interaction was not significant [F(3,78)=1.57, p>0.05], planned comparisons based on the interaction term showed that 200 and 400 mg/kg carbamathione significantly reduced ethanol intake compared to the vehicle condition in nondependent (CTL) mice (# in FIG. 4).

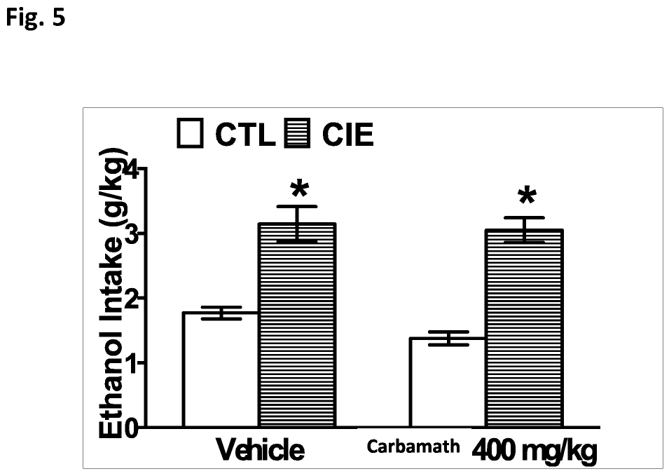

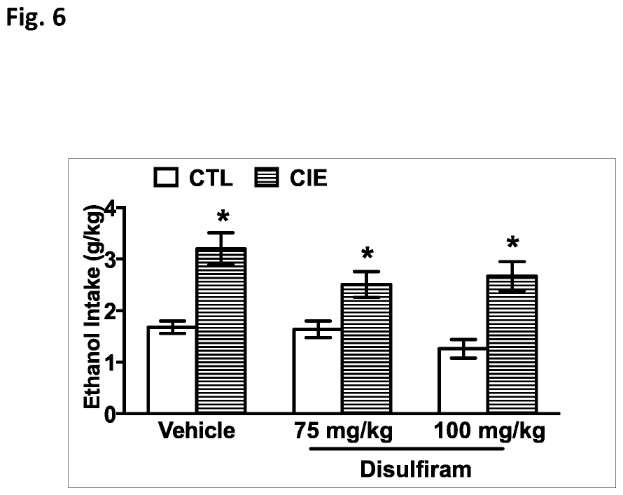

The efficacy of carbamathione in reducing ethanol intake in mice was next compared to the efficacy of disulfiram, an FDA approved drug used in the treatment of chronic alcoholism. The mice were exposed to a sixth cycle of CIE (or air) and evaluated for intake with the same procedure used in the previous test cycle, except that disulfiram was included as a comparator drug. Mice that received vehicle or 400 mg/kg carbamathione continued with this treatment schedule. Mice that received 100 or 200 mg/kg carbamathione were combined and randomly redistributed to receive 75 or 100 mg/kg disulfiram during the first two days and these doses were increased to 125 and 150 mg/kg disulfiram, respectively for the last three days of Test 6. Disulfiram doses were prepared with the same vehicle used for carbamathione (i.e., 0.25% CMC). Separate analyses were conducted to evaluate the effect of carbamathione and disulfiram treatment. Data presented in FIG. 5 show the weekly average intake for CIE and CTL mice that received vehicle or carbamathione. The analysis of these data indicated a significant main effect of Group [F(1,38)=75.22; p<0.0001], with CIE mice drinking more than CTL mice (* in FIG. 5). ANOVA failed to indicate a main effect of carbamathione treatment [F(1,38)=2.28; p>0.05] or a significant group by carbamathione dose interaction [F(1,38)=1.03; p<0.05]. Pair-wise comparisons based on the interaction term indicated a trend for lower ethanol intake in mice treated with 400 mg/kg carbamathione compared to vehicle-treated mice (p=0.07). Data for mice that received vehicle, 75, or 100 mg/kg of disulfiram were averaged over the first two days of the week. Analysis of these data indicated a significant main effect of group [F(1,60)=50.44; p<0.0001], with CIE mice consuming significantly more ethanol that CTL mice (* in FIG. 6). Analysis of these data did not indicate a significant effect of Disulfiram treatment [F(2,60)=2.54; p=>0.05] or an interaction between Group and Disulfiram treatment [F(2,60)=1.26; p>0.05].

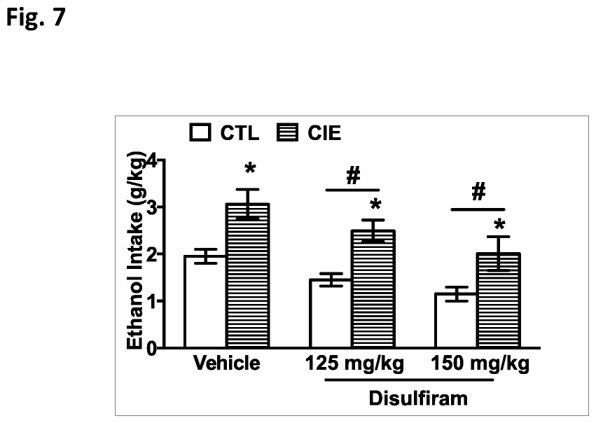

Data for the last three days of Test 6 were average for mice that received vehicle, 125, or 150 mg/kg of disulfiram. ANOVA of these data indicated a significant main effect of Group [F(1,59)=31.00; p<0.0001], with CIE mice consuming more ethanol than CTL mice (* in FIG. 7). There was also a main effect of disulfiram treatment [F(2,59)=8.84; p<0.0001]. Post-hoc comparisons showed that mice treated with disulfiram (averaged over CIE and CTL conditions) showed lower levels of ethanol intake compared to vehicle-treated mice (# in FIG. 7). ANOVA did not indicate a significant interaction between Group and disulfiram treatment [F(2,59)=0.17; p>0.05].

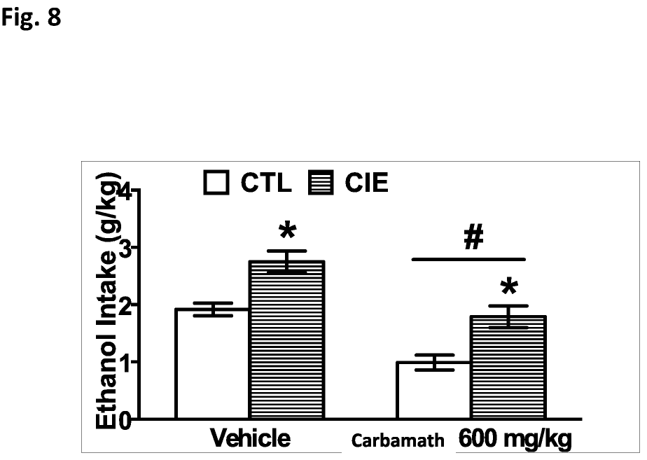

Mice were evaluated again for voluntary ethanol intake after a seventh and final CIE or air exposure cycle. During the five days of Test 7, mice that received vehicle injections from the start of the study continued to receive vehicle injections. Mice that received carbamathione and disulfiram in Test cycles 5 and 6 received vehicle injections in Test 7 to evaluate any long-lasting effect of previous treatment (drug washout evaluation). Finally, mice that received 400 mg/kg carbamathione continued treatment with a higher dose of carbamathione (600 mg/kg). Analysis of the groups that received vehicle injections during Test 7 was performed with Group (CIE, CTL) and previous treatment (vehicle, low, or high disulfiram dose) as main factors. This analysis indicated a significant main effect of Group [F(1,59)=25.36; p<0.0001]. This was due to a significantly higher level of intake in CIE mice compared to CTL mice. ANOVA did not indicate any long-lasting effect of previous drug treatment [F(2,59)=1.06; p>0.05] or a Group x treatment interaction [F(2,59)=0.17; p>0.05] (data not shown). A separate analysis was conducted to evaluate the effect of carbamathione (600 mg/kg) treatment on ethanol drinking in CIE and CTL groups. This analysis indicated significant main effects of Group [F(1,38)=28.43; p<0.0001] and carbamathione dose [F(1,38)=38.88; p<0.0001], but the Group x carbamathione dose interaction was not significant [F(1,38)=0.01; p>0.05]. Post-hoc comparisons indicated that CIE mice consumed more ethanol compared to CTL mice (* in FIG. 8) and carbamathione (600 mg/kg) treatment significantly reduced ethanol intake compared to vehicle-treated subjects in CIE and CTL groups (# in FIG. 8).

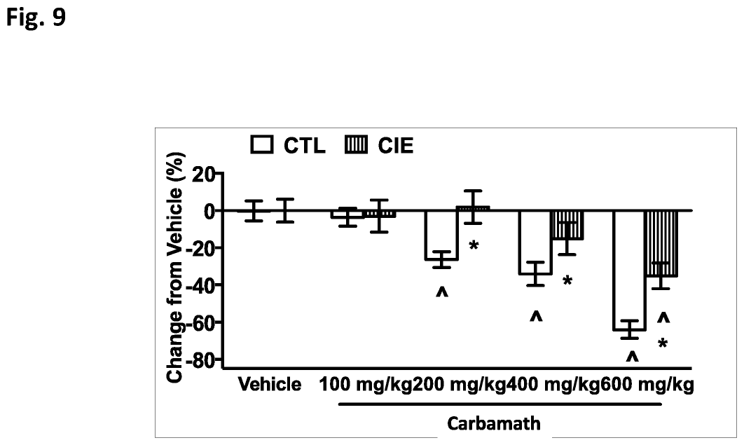

Finally, results obtained during Test cycles 5 and 7 were re-analyzed with data expressed as percent change from the corresponding CIE or CTL vehicle-injected group for mice that received treatment with 100, 200, 400, or 600 mg/kg doses of carbamathione. ANOVA indicated significant main effects of Group [F(1,96)=14.24; p<0.001], carbamathione dose [F(4,96)=18.91; p<0.0001], and a significant interaction between these factors [F(4,96)=2.47; p<0.05]. Post-hoc comparisons based on the interaction term indicated that CTL mice treated with 200, 400, and 600 mg/kg doses of carbamathione showed a significant reduction in voluntary ethanol intake compared to the corresponding vehicle group (\hat{ } in FIG. 7). Additionally, only the highest dose of carbamathione evaluated in this study (600 mg/kg) produced a significant decrease in ethanol intake in CIE mice compare to vehicle subjects (\hat{ } in FIG. 9). Also, carbamathione (200, 400, and 600 mg/kg doses) produced a significantly larger reduction in ethanol intake in CTL mice compared to CIE-exposed mice (* in FIG. 9).

As expected, ethanol intake escalated over successive CIE exposure cycles in dependent mice while ethanol consumption in nondependent mice remained relatively stable throughout the study (Becker and Lopez, Alcohol Clin Exp Res, Vol. 28, No. 12, 2004, pp 1829-1838; Griffin et al., Alcohol Clin Exp Res, Vol. 33, No. 11, 2009, pp 1893-1900; Lopez and Becker, Psychopharmacology, Vol 181, 2005, pp 688-696). This effect was evident during test cycles in which all animals received vehicle treatment (Tests 1-4) and the higher level of ethanol intake in CIE compared to CTL groups was maintained in vehicle treated subjects in subsequent test cycles (Tests 5-7). During the first test cycle in which carbamathione was examined (Test 5), the drug was found to reduce ethanol intake in nondependent (CTL) mice in a dose-related manner while ethanol consumption was not altered in dependent (CIE) mice. In a subsequent test cycle, a higher dose of carbamathione (600 mg/kg) was shown to significantly reduce ethanol intake in CIE-exposed mice as well as CTL mice. Disulfiram was also evaluated to compare its effect to carbamathione. Disulfiram, at 125 and 150 mg/kg doses, reduced ethanol intake in both CIE and CTL subjects. This effect was no longer observed when all subjects received vehicle treatment during a subsequent test cycle (washout test). Finally, analysis of data expressed as percent change from vehicle across test cycles confirmed that carbamathione treatment was relatively more effective in reducing ethanol intake in nondependent subjects than dependent (CIE) subjects. Only the highest dose of carbamathione evaluated (600 mg/kg) produced a significant decrease in ethanol intake in ethanol dependent mice. Taken together, these results suggest that carbamathione significantly reduces voluntary ethanol intake in ethanol dependent and nondependent mice in a dose-related manner. Further, carbamathione appears relatively more effective in reducing ethanol intake in nondependent subjects compared to ethanol dependent subjects.

These data demonstrate that the vehicle used in the carbamathione injections has an influence on the efficacy of carbamathione towards the reduction in ethanol consumption. Without wishing to be bound by theory, it is possible that this difference is due to Tween® 80 interfering with carbamathione absorption once administered to a subject. Further, the observed dose dependency of the carbamathione treatment in this study may be due to the poor solubility of carbamathione. Thus, the use of salt forms of carbamathione may further improve the efficacy of the treatment.

### Example 2. Synthesis and Characterization of Carbamathione (TNX-1001-SM)

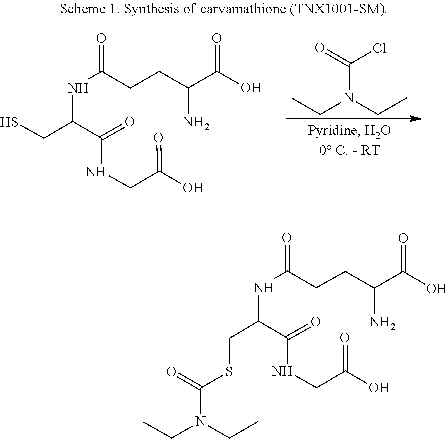

Glutathione (9.0 g, 29.28 mmol) was weighed and transferred into a 1 L-round-bottom flask equipped with a magnetic stirring bar. H₂O (100 mL) and pyridine (200 mL) were added and the complete dissolution of the starting material was observed. The mixture was cooled to 0° C. in an ice-bath and stirred at this temperature for 30 minutes.

Diethyl carbamoyl chloride (11.1 mL, 87.84 mmol) in pyridine (80 mL) was transferred into a dropping funnel and slowly added to the reaction (approximately 2 hours). The ice-water bath was removed and the reaction mixture was stirred at room-temperature overnight. The solvent was removed completely by rotavap (bath temp. 60° C., 100 mbar) to give a pale yellow waxy solid. A H₂O/EtOH mixture (5/95, 800 mL) was added and the reaction was stirred at room temperature for 2 hours and then stored in the fridge (4° C.) overnight.

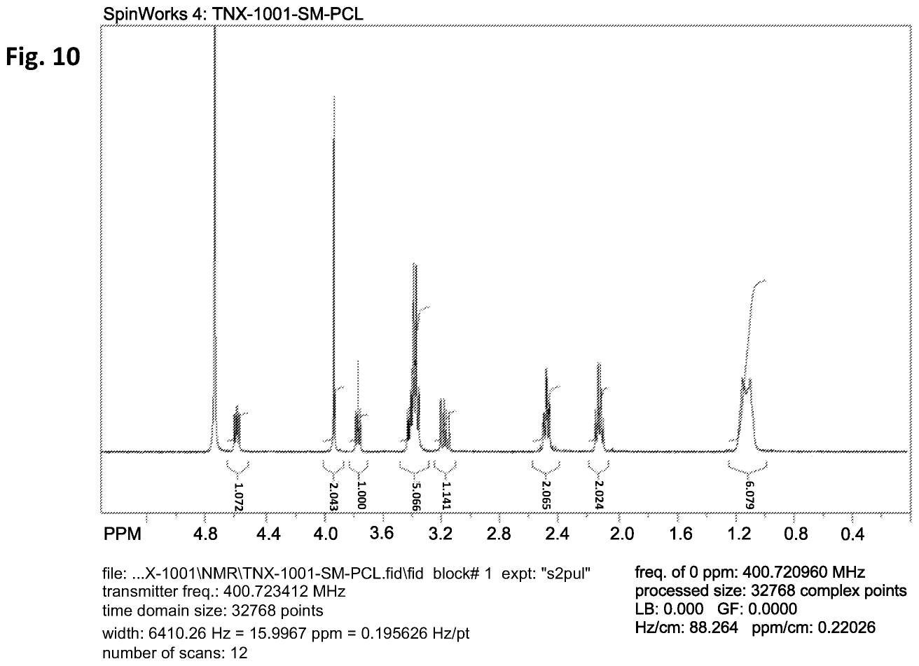

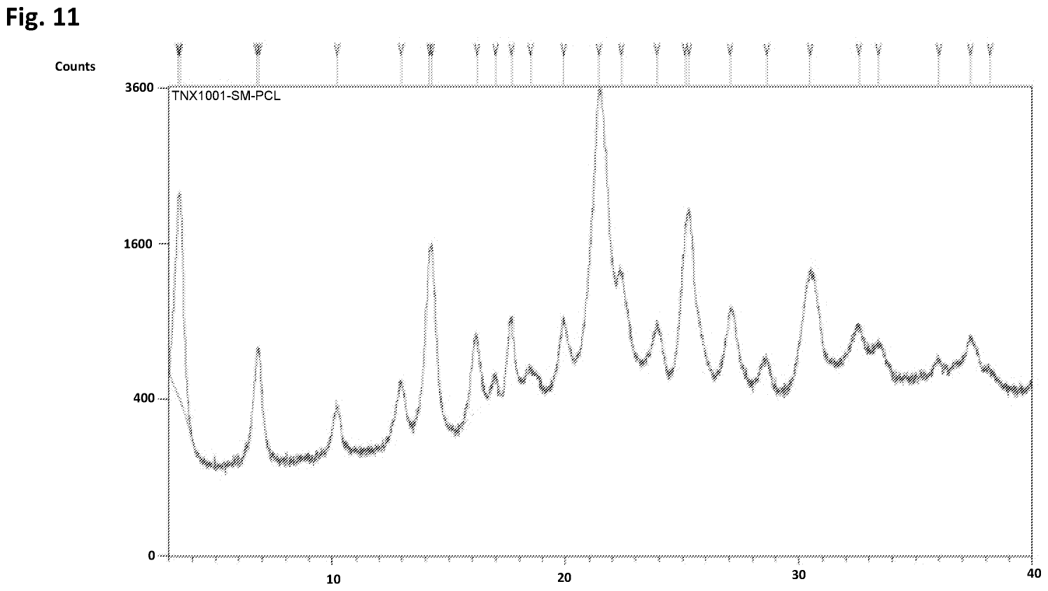

The formed precipitate was recovered by vacuum filtration, washed with cold Ethanol (100 mL) and dried at 40° C. and 50 mbar overnight. 3.46 g of white solid was recovered (yield=29%). ¹H NMR (400 MHz, D₂O): δ 4.60 (dd, 1H, J=5.0, 8.2 Hz), 3.94 (s, 2H), 3.7 (t, 1H, J=6.4 Hz), 3.32-3.46 (m, 5H), 3.18 (dd, 1H, J=8.2, 14.4 Hz), 2.42-2.56 (m, 2H), 2.12 (quart., 2H, J=7.7 Hz), 1.04-1.20 (m, 6H). See FIG. 10 for the ¹H NMR spectrum. The sample was also characterized by XRPD (FIG. 11). The XRPD peaks for TNX1001-SM are listed Table 3 below.

TABLE 3
Carbamathione (TNX1001-SM) XRPD characterization

| | | | d-spacing | |
|---|---|---|---|---|
| Pos. [° 2Th.] | Height [cts] | FWHM [° 2Th.] | [Å] | Rel. Int. [%] |
| 3.3877 | 1642.67 | 0.0689 | 26.08139 | 56.44 |
| 3.4581 | 1747.16 | 0.0886 | 25.55060 | 60.03 |
| 6.7697 | 539,.9 | 0.0984 | 13.05732 | 18.55 |
| 6.8697 | 529.94 | 0.0590 | 12.86752 | 18.21 |
| 10.1815 | 192.13 | 0.3149 | 8.68820 | 6.60 |
| 12.9393 | 248.15 | 0.2755 | 6.84203 | 8.53 |
| 14.1599 | 1274.83 | 0.0787 | 6.25486 | 43.81 |
| 14.2685 | 1300.27 | 0.1574 | 6.20749 | 44.68 |
| 16.1982 | 447.51 | 0.1378 | 5.47208 | 15.38 |
| 16.9798 | 118.49 | 0.2362 | 5.22192 | 4.07 |
| 17.6695 | 466.26 | 0.1771 | 5.01960 | 16.02 |
| 18.4799 | 123.27 | 0.2362 | 4.80126 | 4.24 |
| 19.9140 | 382.12 | 0.3149 | 4.45862 | 13.13 |
| 21.4142 | 2910.24 | 0.1968 | 4.14955 | 100.00 |
| 22.3939 | 683.10 | 0.2362 | 3.97017 | 23.47 |
| 23.9001 | 306.26 | 0.3936 | 3.72327 | 10.52 |
| 25.1168 | 1228.50 | 0.0984 | 3.54561 | 42.21 |
| 25.2933 | 1393.40 | 0.0984 | 3.52126 | 47.88 |
| 27.0480 | 463.14 | 0.3149 | 3.29668 | 15.91 |
| 28.6205 | 164.16 | 0.3936 | 3.11903 | 5.64 |
| 30.4646 | 780.45 | 0.5510 | 2.93430 | 26.82 |
| 32.5571 | 270.13 | 0.3149 | 2.75033 | 9.28 |
| 33.3992 | 183.77 | 0.2755 | 2.68288 | 6.31 |
| 35.9849 | 96.71 | 0.1574 | 2.49582 | 3.32 |
| 37.3424 | 236.82 | 0.1574 | 2.40815 | 8.14 |
| 38.1741 | 60.21 | 0.3936 | 2.35758 | 2.07 |

### DSC/TGA

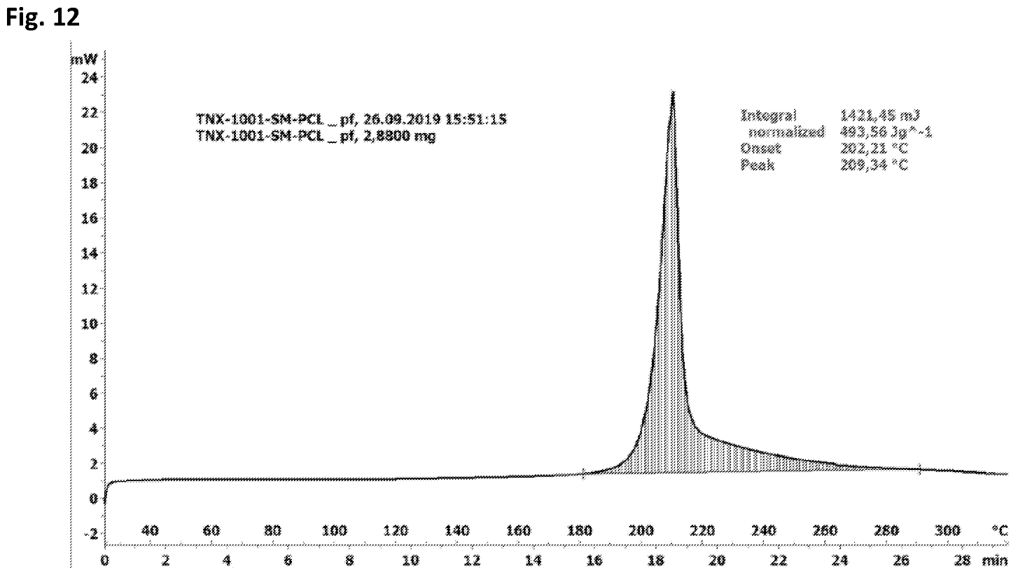

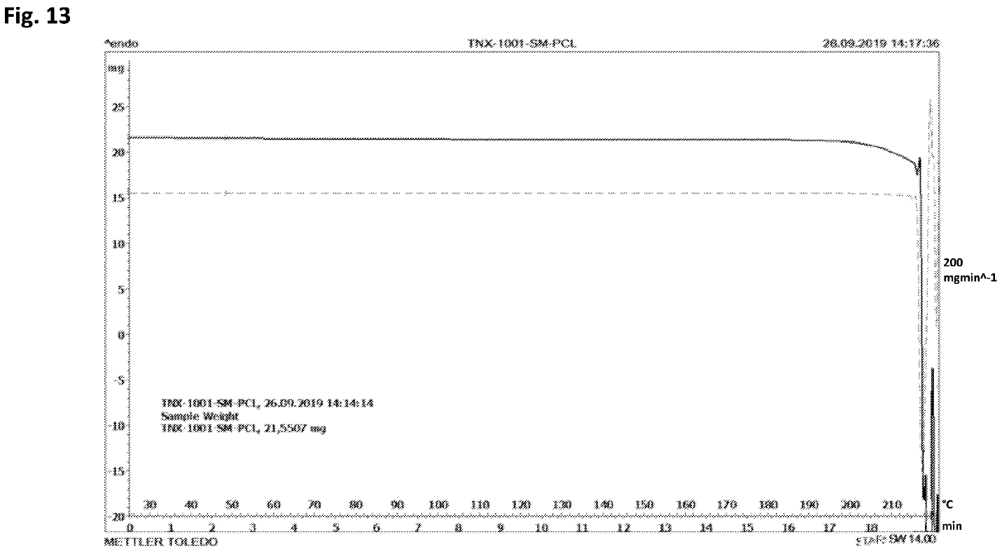

DSC analysis of TNX1001-SM exhibits an endothermic event at 209.3° C. (onset 202.2° C.) imputable to melting and decomposition of the product (FIG. 12). The TGA profile is typical of an anhydrous compound decomposing above 200° C. (FIG. 13). Evolved Gas Analysis (EGA) was consistent with loss of carbonyl sulfide.

### FT-IR

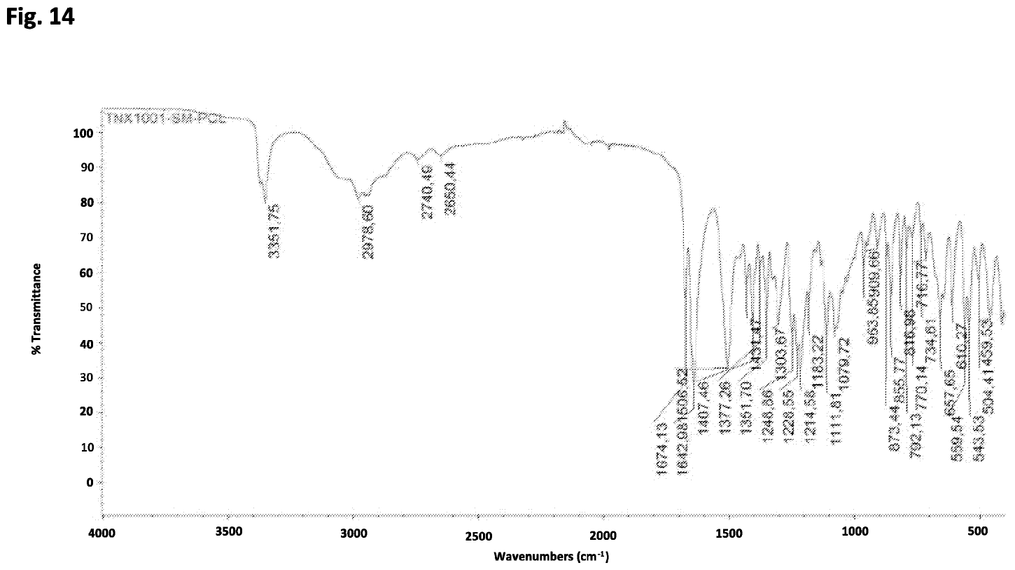

The FT-IR spectrum of carbamathione (TNX1001-SM) is shown in FIG. 14. The corresponding peaks are provided in Table 4 below.

TABLE 4
FT-IR peak list of carbamathione (TNX1001-SM)

| Position | Intensity |
|---|---|
| 410.91 | 45.075 |
| 459.53 | 47.139 |
| 504.41 | 57.853 |
| 543.53 | 45.214 |
| 559.54 | 46.269 |
| 610.27 | 50.226 |
| 657.65 | 46.488 |
| 716.77 | 63.976 |
| 734.61 | 70.790 |
| 770.14 | 69.587 |
| 792.13 | 66.503 |
| 816.98 | 58.642 |
| 855.77 | 53.447 |
| 873.44 | 63.486 |
| 909.66 | 67.599 |
| 963.85 | 61.692 |
| 1079.72 | 42.072 |
| 1111.81 | 41.496 |
| 1183.22 | 51.498 |
| 1214.58 | 32.647 |
| 1228.55 | 37.277 |
| 1248.86 | 42.925 |
| 1303.67 | 45.760 |
| 1351.70 | 48.200 |
| 1377.26 | 62.145 |
| 1407.46 | 46.472 |
| 1431.47 | 56.564 |
| 1506.52 | 33.219 |
| 1642.98 | 28.613 |
| 1674.13 | 52.341 |
| 2650.44 | 93.204 |
| 2740.49 | 92.204 |
| 2978.60 | 80.897 |
| 3351.75 | 80.700 |

### Example 3. Salt/Co-Crystal Screening

A salt/co-crystal screening was carried out for carbamathione. Solid or liquid based methods were used to screen for the formation of salts/co-crystals, including solid state grinding/kneading, slurry maturation, solution crystallization (crystallization from a saturated solution and precipitation) and solvent evaporation. The formation of a salt was assessed with various co-formers including, L-lysine, NaOH, p-toluenesulfonic acid monohydrate, sulfuric acid, and methanesulfonic acid. Those skilled in the art will recognize that other co-formers can also be tested, including, but not limited to, benzenesulfonic acid, cyclamic acid, ethanedisulfonic acid, ethanesulfonic acid, 1-naphthalenesulfonic acid, 2-naphthalenesulfonic acid, L-arginine, deanol, choline, and diethylamine, N-cyclohexylsulfamic acid, camphor-10-sulfonic acid, naphthalenedisulfonic acid, quinaldic acid, and those summarized in Table 5.

TABLE 5
List of selected co-formers for the salt/co-crystal screening.

| | | MW | | | GRAS |
|---|---|---|---|---|---|
| ID | Co-Former | (g/mol) | pKa | M.P (° C.) | Status |
| ACA | Acetic Acid | 60.05 | 4.76 | 16 | 1 |
| ADI | Adipic Acid | 146.14 | 4.44 | 151 | 1 |
| BEN | Benzoic Acid | 122.123 | 4.20 | 122.4 | 1 |
| CAM | (+)-Camphoric Acid | 200.23 | 4.72 | 183 | 2 |
| CIA | Citric Acid | 192.12 | 3.1 | 153 | 1 |
| FUA | Fumaric Acid | 116.08 | 3.03 | 287 | 1 |
| GTR | Glutaric Acid | 132.11 | 4.34 | 95.8-98 | 1 |
| GLY | Glycolic Acid | 76.05 | 3.28 | 75 | 1 |
| LTA | L-Tartaric Acid | 150.09 | 3.02 | 170 | 1 |
| MAI | DL-Malic Acid | 134.09 | 1.92 | 131 | 1 |
| MEA | Maleic Acid | 116/07 | 1.92 | 131 | 1 |
| OXA | Oxalic Acid | 90.03 | 1.27 | 189 | 2 |
| PHA | Phosphoric Acid | 97.994 | 2.15 | / | 1 |
| | | | 7.20 | | |
| | | | 12.35 | | |
| SOR | Sorbic Acid | 112.13 | 4.76 | 132 | 1 |
| SUA | Succinic Acid | 118.09 | 4.21 | 185 | 1 |
| KOH | Potassium Hydroxide | 56.11 | 0.5 | 361 | 1 |
| LLYS | L-Lysine | 146.19 | 10.79 | 215 | 1 |
| MEG | Meglumine | 195.21 | 9.50 | 129 | 1 |
| PYX | Pyridoxine | 169.18 | 5.58 | 159 | 1 |
| TRO | Tromethamine | 121.14 | 8.07 | 171 | 2 |

GRAS: Generally Recognized as Safe.
M.P.: Melting point

### Solubility of Free Carbamathione (TNX1001-SM)

Initially, the solubility of free carbamathione was evaluated in water and in common organic solvents. The common organic solvents tested included dichloromethane (DCM), methanol, ethyl acetate, ethanol, acetonitrile, acetone, 2-propanol, and N,N-dimethylformamide.

For each solvent the solubility of carbamathione was assessed by weighing out 50 mg of carbamathione into a stoppered tube followed by the addition of 0.05 mL of the test solvent to the tube. The mixture underwent vigorous shaking for 1 minute, and was placed in a constant temperature device for 15 minutes at 25.0±0.5° C. If the carbamathione was not completely dissolved, vigorous shaking was repeated for 1 minute and then placed in a constant temperature device for an additional 15 minutes. If the carbamathione was not fully dissolved, additional solvent was added portion-wise until dissolution of carbamathione was observed. If complete dissolution was not observed, the solution was heated until the boiling point under stirring to verify the solubility at high temperature. The solvents were classified according to the visual solubility determined into the groups described in Table 6.

TABLE 6
Solubility rages description

| | | Parts of solvent needed | Solubility |
|---|---|---|---|
| Descriptive Terms | Abbreviation | for 1 part solute | (mg/mL) |
| Very soluble | VS | <1 | >1000 |
| Freely Soluble | FS | 1-10 | 100-1000 |
| Soluble | S | 10-30 | 33-100 |
| Sparingly Soluble | SS | 30-100 | 10-33 |
| Slightly Soluble | VSS | 100-1000 | 1-10 |
| Very Slightly | | 1000-10000 | 1-0.1 |
| soluble | | | |
| Insoluble | INS | >1000 | <0.1 |

It was found that carbamathione was very slightly soluble in all of the common organic solvents and sparingly soluble in water only at high temperature.

Several solvents were selected in order to vary as much as possible the crystallization medium properties in terms of solvent class, polarity, boiling point and hydrogen bond acceptor/donor propensity while also considering the solubility properties of the starting material.

Tert-Butyl methyl ether (TBME) was employed as anti-solvent in some slurry experiments. The main physical-chemical properties of the employed solvents and the results of the solubility tests are listed in the Table 7 below.

TABLE 7
Results of Solubility testing

| | | ICH | BP | MP | | Solubility |
|---|---|---|---|---|---|---|
| ID | Solvent | Class | (° C.) | (° C.) | Solubility | at HT |
| 2PR | 2-Propanol | 3 | 82.4 | −88.5 | VSS (<10 | No |
| | | | | | mg/mL) | |
| ACN | Acetonitrile | 2 | 82 | −44 | VSS (<10 | No |
| | | | | | mg/mL) | |
| ACT | Acetone | 3 | 56.2 | −94.3 | VSS (<10 | No |
| | | | | | mg/mL) | |
| DCM | Dichloromethane | 2 | 40 | −97 | VSS (<10 | No |
| | | | | | mg/mL) | |
| DMF | N,N- | 2 | 153 | −61 | VSS (<10 | No |
| | Dimethylformamide | | | | mg/mL) | |
| ETA | Ethyl Acetate | 3 | 77 | −83.6 | VSS (<10 | No |
| | | | | | mg/mL) | |
| ETH | Ethanol | 3 | 78.5 | −114.1 | VSS (<10 | No |
| | | | | | mg/mL) | |
| H2O | Water | / | 100 | 0 | VSS (<10 | Dissolved |
| | | | | | mg/mL) | (100° C.) |
| MET | Methanol | 2 | 64.6 | −98 | VSS (<10 | No |
| | | | | | mg/mL) | |
| THF | Tetrahydrofuran | 2 | 66 | −108.4 | VSS (<10 | No |
| | | | | | mg/mL) | |

All the mixtures employed for solubility evaluation were stirred for 3 days at room temperature except for the mixture in water. The recovered solids were analyzed by XRPD to investigate the presence of potential polymorphs and/or solvates of carbamathione that might be encountered during the study. All of the analyzed solids displayed the same diffractogram as the carbamathione starting material.

By cooling the hot aqueous solution, a few milligrams of solid were recovered and analyzed; at the same time the filtrate solution was evaporated at high temperature (60° C.), and the solid obtained was analyzed by XRPD. Both the solids displayed a diffractogram superimposable with that of the carbamathione starting material

### Slurry Experiment in Water

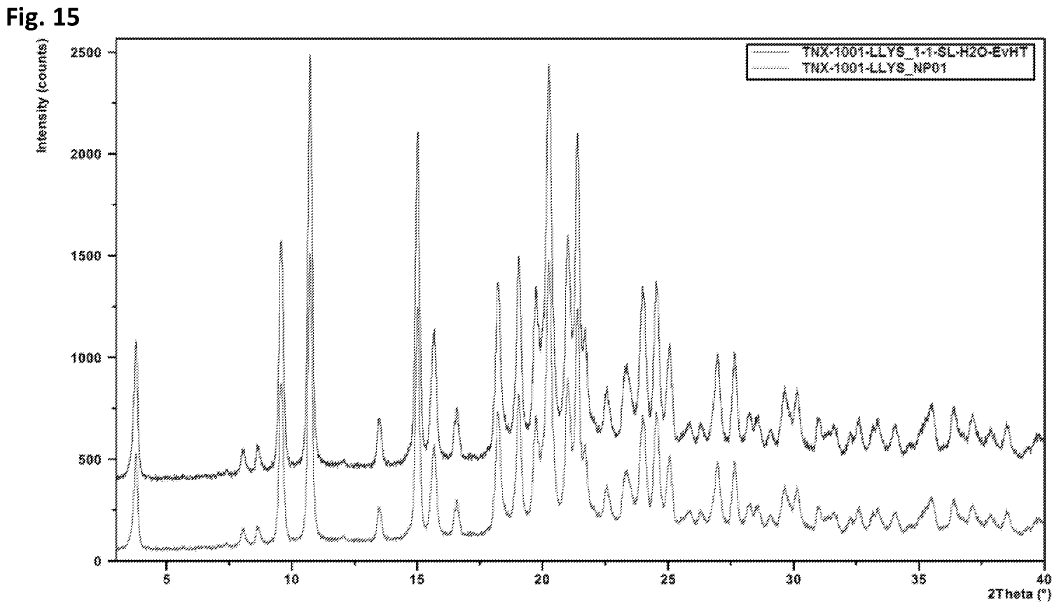

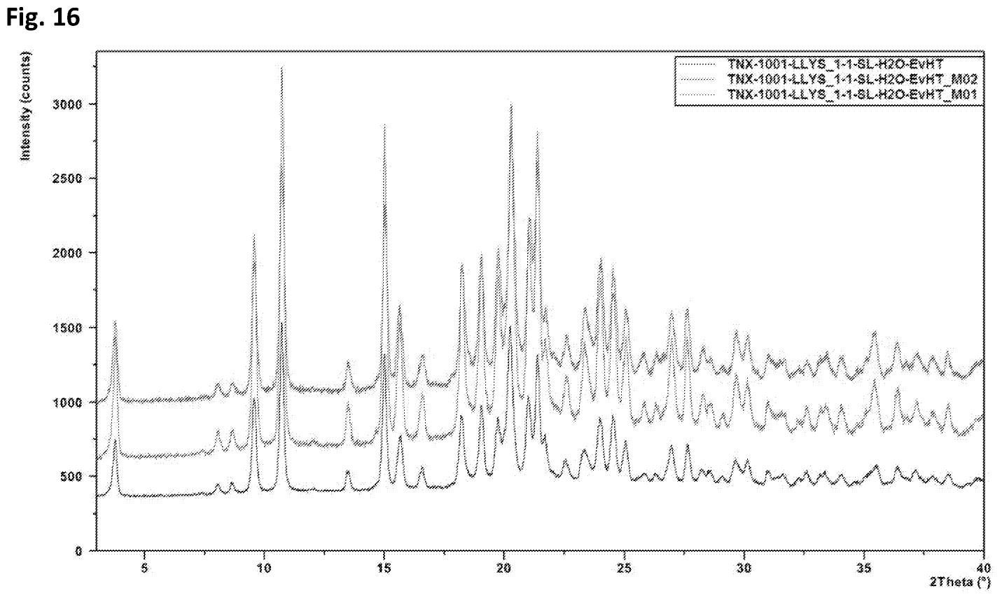

Carbamathione (50 mg) and one equivalent of L-Lysine were weighed in an 8-mL glass vial. Water (1-2 mL) was added and the mixture was allowed to stir for 24 hours. The equimolar mixture of carbamathione and L-Lysine was soluble in water and no precipitate was observed after 24 hours of stirring. The solution was left to evaporate at high temperature (60° C.) and an off-white solid was isolated. XRPD analysis confirmed the recovery of new derivative TNX1001-LLYS-NP01 (FIG. 15). The sample was analyzed after 24 hours and after 4 days and the comparison between the diffractograms highlighted a good stability of the sample under ambient conditions (FIG. 16).

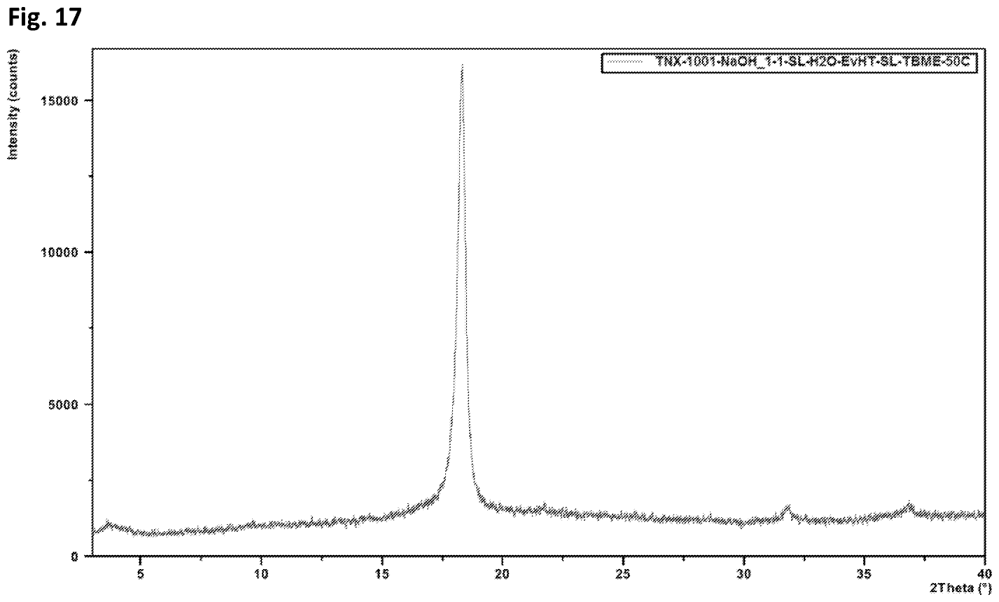

The slurry experiment in water was repeated with NaOH as the co-former. Carbamathione (50 mg) and one equivalent of NaOH were weighed in an 8-mL glass vial. Water (1-2 mL) was added and the mixture was allowed to stir for 24 hours resulting in a clear solution. The liquid was left to evaporate resulting in the formation of a sticky solid/oil that was further slurried in TBME at 50° C. for 3 days. The white solid obtained from the slurry experiment carried out with NaOH as co-former was analyzed by XRPD which revealed the formation of an amorphous phase (FIG. 17).

### Slurry Experiment in Methanol

The slurry experiment was repeated in methanol. Carbamathione (TNX1001-SM) (50 mg) and one equivalent of L-lysine were weighed in an 8-mL glass vial equipped with a magnetic stirring bar. Methanol (1-2 mL) was added and the mixture was left stirring at room temperature for approximately 24 hours.

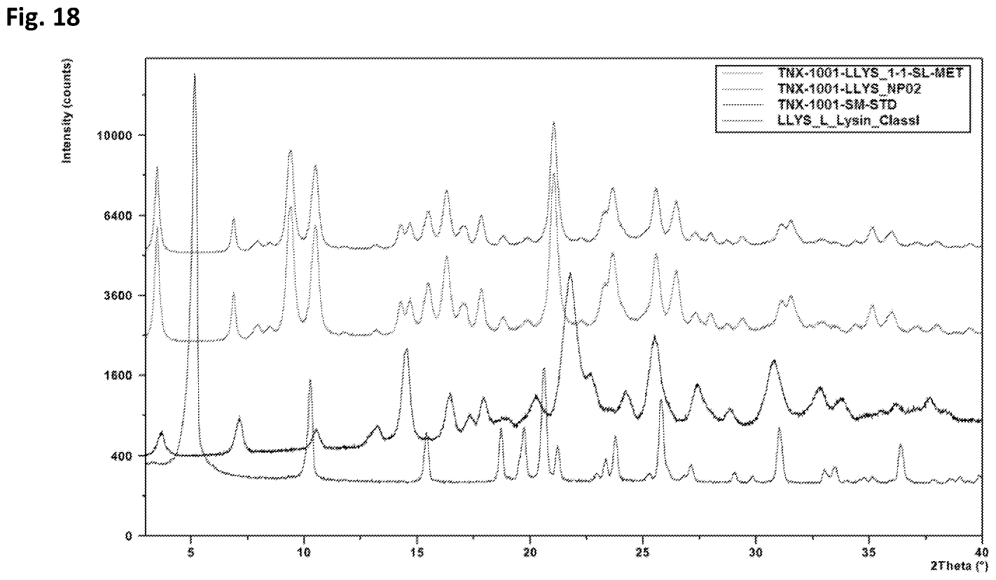

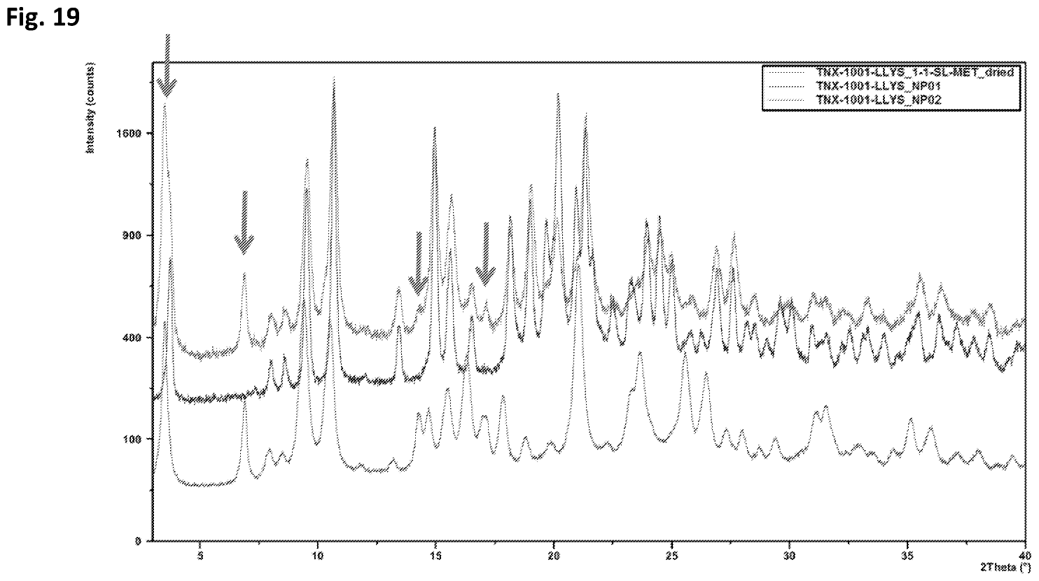

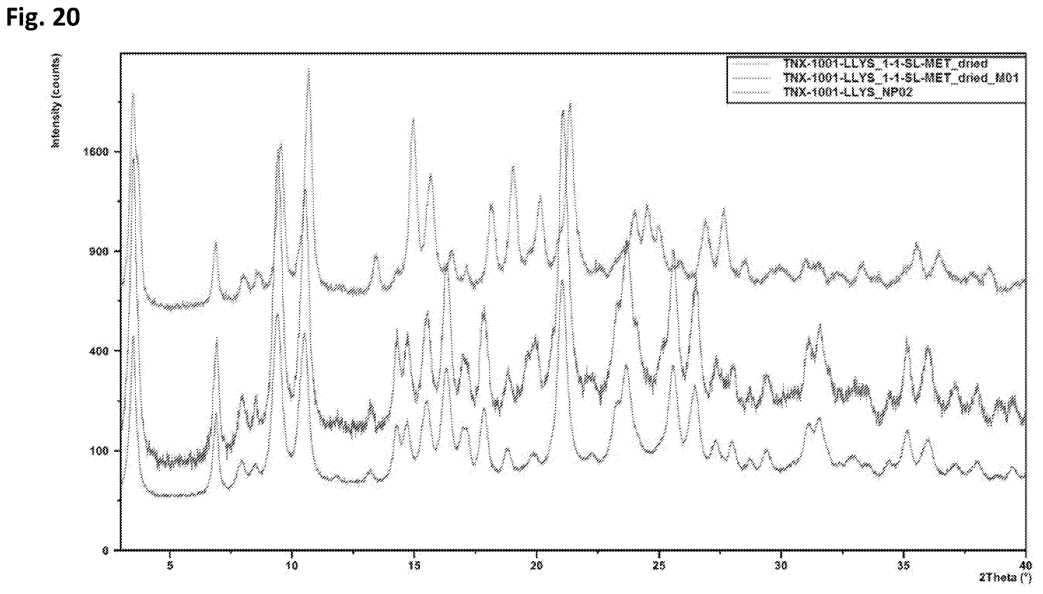

After 24 hours of stirring, the solid was collected and analyzed by XRPD, and a new diffraction pattern was observed (FIG. 18). This new pattern was labeled as TNX1001-LLYS-NP02. The solid was dried for 18 hours at 40° C. under vacuum (50 mbar) and XRPD analysis of the dried sample showed a diffractogram compatible with the presence of the derivative TNX1001-LLYS-NP01 (see the slurry experiment in water), although some residual peaks imputable to the presence of TNX1001-LLYS-NP02 were still visible (highlighted by arrows in FIG. 19). After the drying step, the sample was exposed to humidity for 24 hours and the diffractogram acquired again. As shown in FIG. 20, the sample converted spontaneously to initial form TNX1001-LLYS-NP02.

### Slurry Experiment in Dichloromethane

The slurry experiment was repeated in dichloromethane (DCM). Carbamathione (50 mg) and one equivalent of L-lysine were weighed in an 8-mL glass vial. DCM (1-2 mL) was added and the mixture was left under stirring 1 day at room temperature.

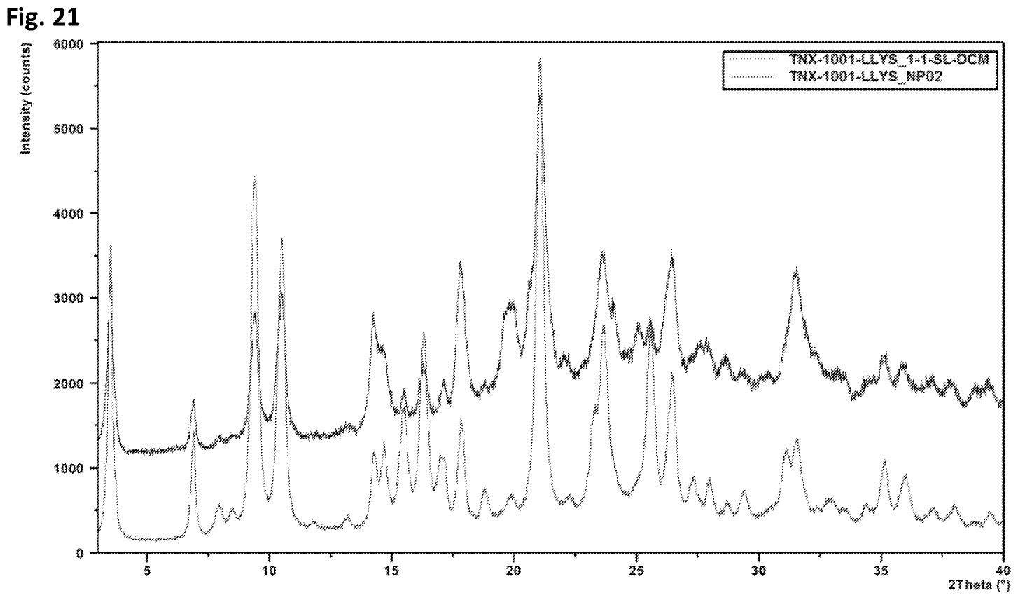

After 24 hours of stirring, the solid was collected and analyzed by XRPD. The new derivative that was observed in the methanol slurry experiments, TNX1001-LLYS-NP02, as recovered (FIG. 21).

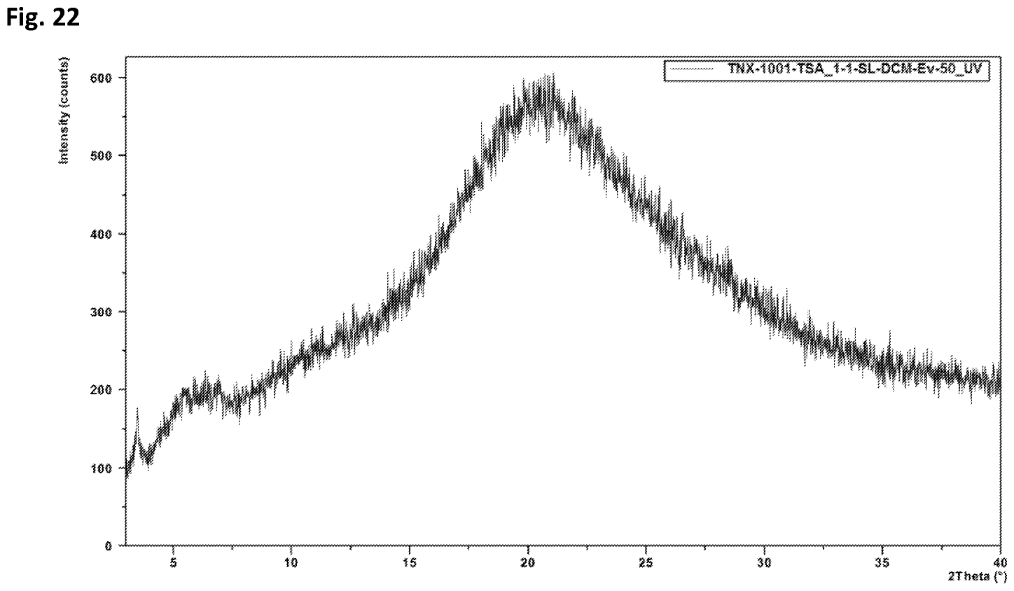

The dichloromethane slurry experiment was repeated using p-toluenesulfonic acid monohydrate (TSA) as the co-former. Carbamathione (50 mg) and one equivalent of TSA were weighed in an 8-mL glass vial. DCM (1-2 mL) was added and the mixture was left under stirring 1 day at room temperature resulting in a clear solution. The liquid was evaporated at high temperature (60° C.) and a sticky solid was obtained. The sticky solid was further slurried in TBME at 50° C., resulting in the recovery of a white solid. Analysis of the white solid by XRPD revealed the formation of an amorphous phase (FIG. 22).

### Kneading

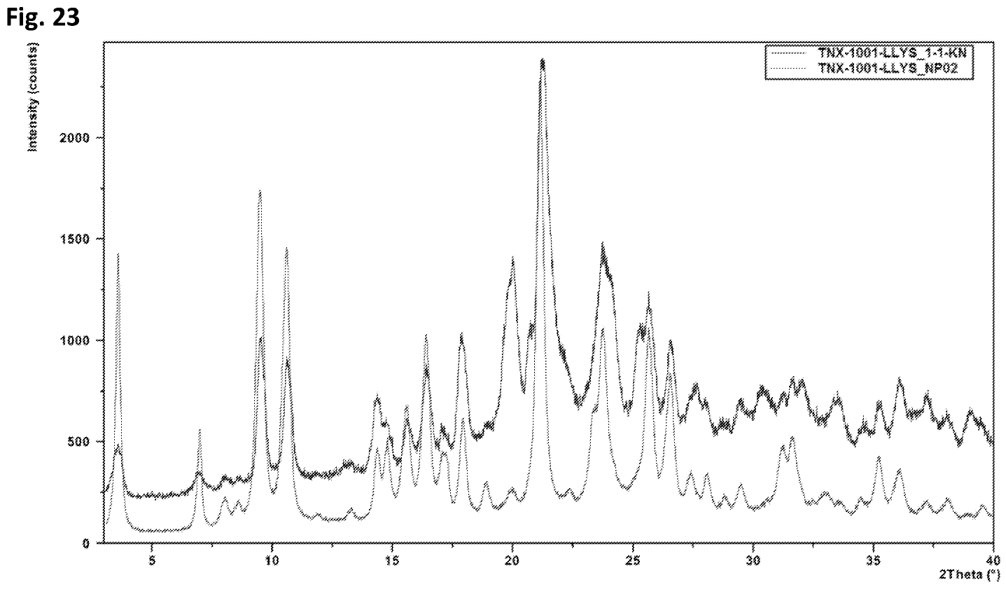

Carbamathione, one equivalent of L-lysine and a catalytic amount of water (10 μL) were ground by ball milling in a Retsch MM 200 grinder for 20 minutes at a frequency of 30 Hz. The solid was then collected and analyzed by XRPD. The resulting diffractogram revealed the recovery of the L-lysine derivative that was previously observed in the slurry experiments with methanol and dichloromethane (TNX1001-LLYS-NP02) (FIG. 23).

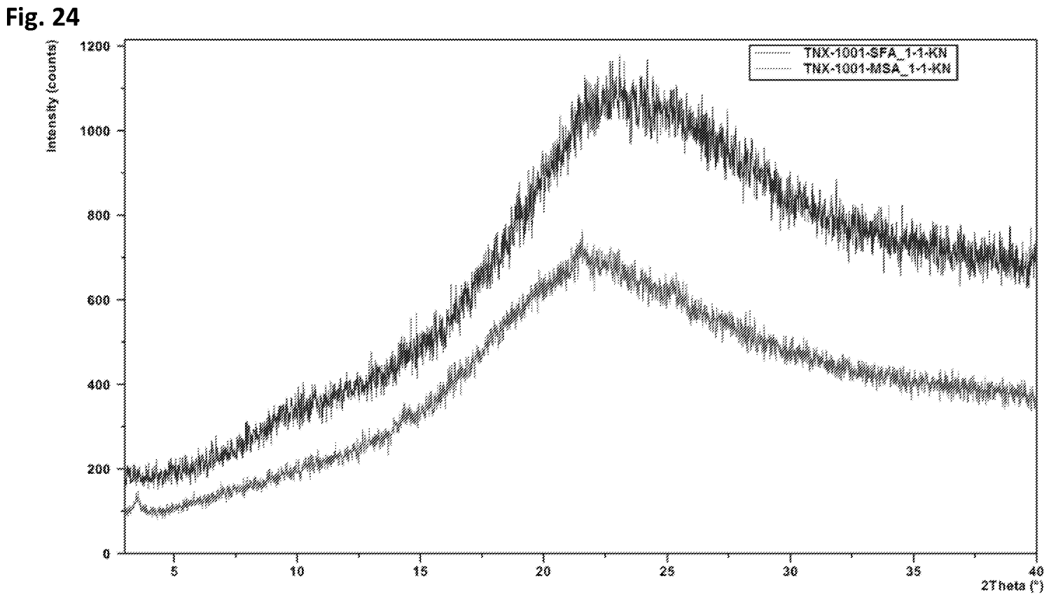

The kneading experiment was repeated independently with one equivalent of sulfuric acid (SFA) and methanesulfonic acid (MSA). From those experiments sticky solids that showed an amorphous XRPD profile were recovered (FIG. 24).

Experiment with HCl as Co-Former

TNX1001-SM (100 mg) was weighed and transferred into a 50-mL round bottom flask equipped with a magnetic stirring bar. Methanol (5 mL) and HCl 37% (1 eq., 20.2 μL) were added and a clear solution was immediately realized. The solvent was removed by rotavap (bath temperature 40° C., 70 mbar) furnishing a sticky oil. Cyclohexane (20 mL) was added to the sticky oil, which was subsequently removed by rotavap. The cyclohexane addition and removal was repeated three times in order to remove any traces of water containing HC137%. Finally the sticky oil was dried by oil pump (0.1 mbar) at room temperature overnight.

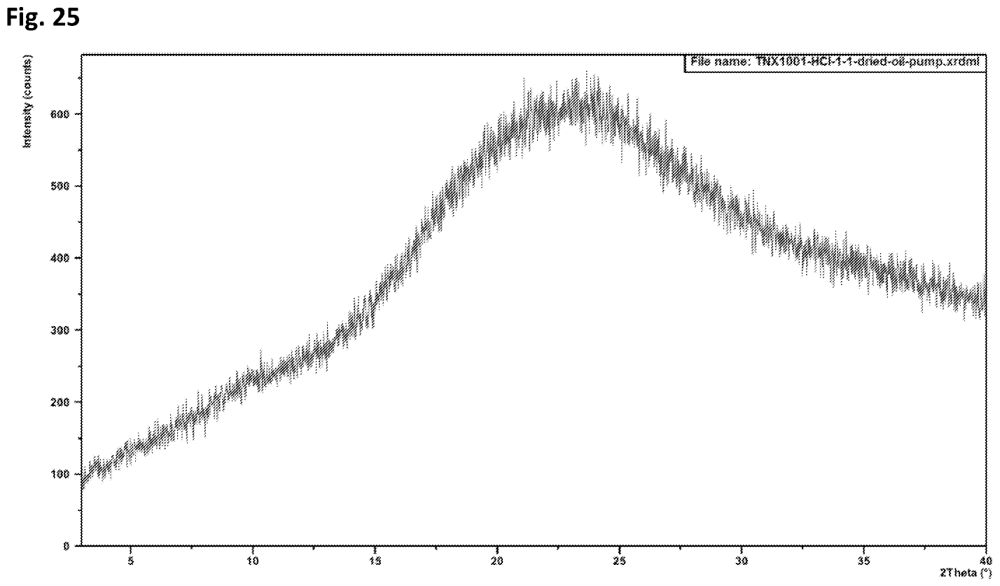

The recovered glassy solid showed a high hygroscopicity and by XRPD analysis the recovery of an amorphous profile was confirmed (FIG. 25).

### SUMMARY OF RESULTS

Two new XRPD patterns associated to TNX1001 and L-lysine adducts were identified and labelled TNX1001-LLYS-NP01 and TNX1001-LLYS-NP02.

Five amorphous materials were obtained from experiments using NaOH, p-toluenesulfonic acid monohydrate, sulfuric acid, methanesulfonic acid and HCl as co-formers.

The new solid phase associated to TNX1001-LLYS-NP01 was recovered by evaporation at high temperature (60° C.) of an aqueous solution of TNX1001-SM and L-lysine in equimolar ratio. The pattern turned out to be stable under ambient conditions up to 4 days since no appreciable differences were observed in the XRPD diffractogram of the sample acquired again after this time.

The experiment was duplicated on 50 mg of starting material and scaled-up to 150 mg confirming the recovery of the new derivative. Sample TNX1001-LLYS-NP01-150 mg was completely characterized (see Characterization of New Patterns below). This adduct displayed a clear improvement in water solubility as compared to free carbamathione.

From the experiments carried out with L-lysine in organic media, a second new diffraction pattern was also observed, in particular when an equimolar mixture of TNX1001-SM and L-lysine co-former was slurried at room temperature for 24 hours in Methanol or Dichloromethane (TNX1001-LLYS-NP02). The sample recovered from the slurry experiment in methanol (TNX1001-LLYS-1-1-SL-MET) was further dried at 50 mbar and 40° C. overnight and the conversion into TNX1001-LLYS-NP01 was observed, although some traces of NP02 were still visible. Exposure of the dried sample to humidity results in reconversion of the NP01 form into the NP02 form after approximately 24 hours, as confirmed by XRPD analysis.

The diffraction pattern attributable to TNX1001-LLYS-NP02 was observed also for the solid recovered after slurry in DCM at room temperature for 24 hours of an equimolar mixture of TNX1001-SM and L-lysine and by kneading with a catalytic amount of water.

The recovery of the same solid form from two different solvents suggests that the product is not a solvated form. Furthermore, the conversion observed by drying followed by reconversion after exposition to humidity confirms this supposition and advises the presence of a hydrate derivative of the new Carbamathione Lysine salt.

### Characterization of New Patterns

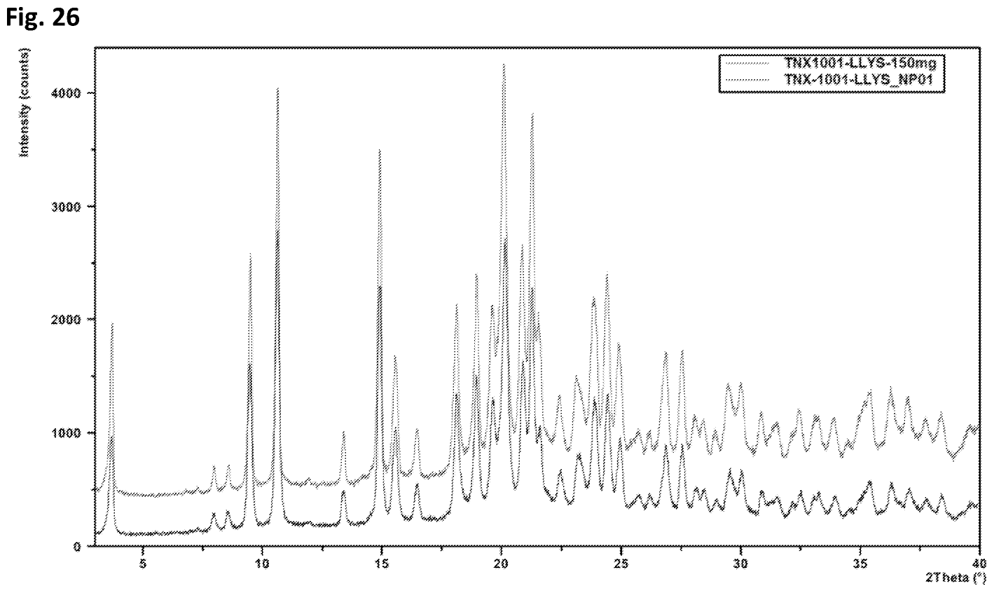

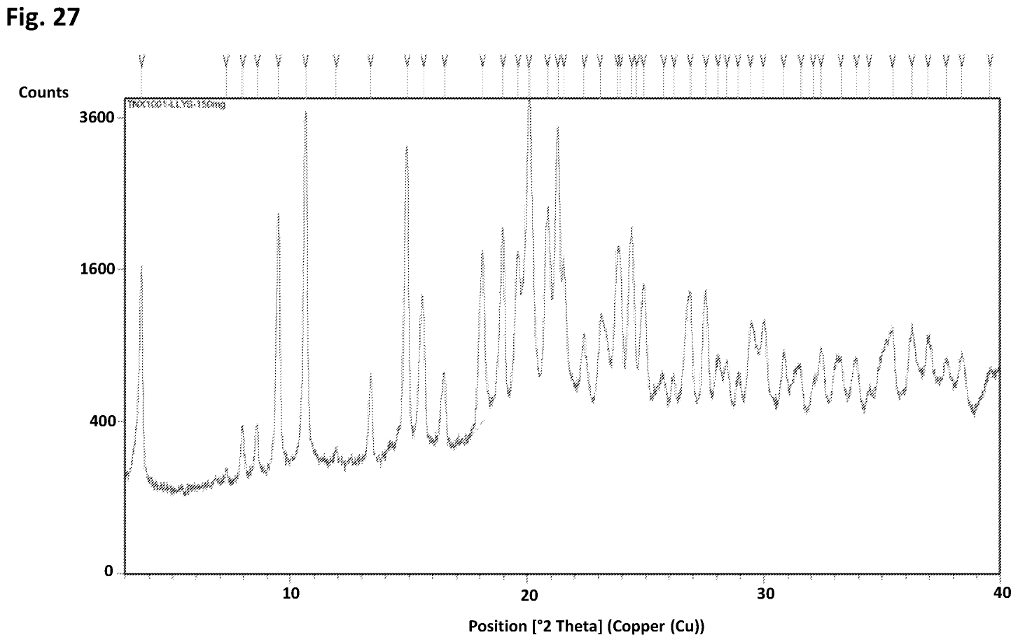

The synthesis of the new derivative, TNX1001-LLYS-NP01, was carried out to facilitate its complete characterization. TNX1001-SM (150 mg) and L-Lysine (1 eq., 54 mg) were accurately weighed in a vial equipped with a magnetic stirring bar. H₂O (3 ml) was added and the mixture was stirred at room temperature for 4 hours until a clear solution was obtained. The solution was filtered through a 0.45 μm RC-filter and the filtrate was evaporated at high temperature (60° C.). The recovered off-white solid was compared with the sample that was obtained from the slurry experiments in water by XRPD analysis to confirm the recovery of desired derivative (FIG. 26). The product was completely characterized using the methods outlined in Table 8 (see FIGS. 27-33). The XPRD peaks are listed in Table 9 below.

TABLE 8
List of characterization methods.

| Analysis | Acronym | Rationale |
|---|---|---|
| X-ray Powder Diffraction | XRPD | For identification of new species |
| Differential Scanning | DSC | For confirmation of new species |
| Calorimetry | | and evaluation of their purity |
| Thermal Gravimetric | TGA/EGA | For identification of solvates or |
| Evolved Gas Analysis | | hydrates |
| Fourier Transform | FT-IR | For confirmation of new species |
| Infrared Spectroscopy | | |
| ¹H Magnetic Resonance | ¹H-NMR | For tentative stoichiometry |
| in Solution | | assignment |

TABLE 9
XRPD peak list for TNX1001-LLYS-NP01

| Pos. | Height | FWHM | d-spacing | Rel. Int. |
|---|---|---|---|---|
| [° 2Th.] | [cts] | [° 2Th.] | [Å] | [%] |
| 3.6959 | 1486.14 | 0.0787 | 23.90700 | 42.62 |
| 7.2787 | 36.71 | 0.0984 | 12.14527 | 1.05 |
| 7.9712 | 196.99 | 0.0984 | 11.09169 | 5.65 |
| 8.6016 | 166.33 | 0.1082 | 10.28013 | 4.77 |
| 9.4909 | 2044.57 | 0.1279 | 9.31879 | 58.64 |
| 10.6341 | 3486.73 | 0.1082 | 8.31942 | 100.00 |
| 11.9148 | 56.18 | 0.1574 | 7.42795 | 1.61 |
| 13.3703 | 460.56 | 0.0492 | 6.62241 | 13.21 |
| 14.9275 | 2660.30 | 0.1181 | 5.93489 | 76.30 |
| 15.6202 | 873.10 | 0.0590 | 5.67324 | 25.04 |
| 16.5015 | 370.56 | 0.0492 | 5.37218 | 10.63 |
| 18.0999 | 1394.85 | 0.0689 | 4.90120 | 40.00 |
| 18.9789 | 1517.58 | 0.0984 | 4.67613 | 43.52 |
| 19.5970 | 1226.81 | 0.1771 | 4.53003 | 35.19 |
| 20.0613 | 3274.50 | 0.1200 | 4.42256 | 93.91 |
| 20.1184 | 3246.01 | 0.0480 | 4.42110 | 93.10 |
| 20.8543 | 1692.57 | 0.0600 | 4.25614 | 48.54 |
| 21.2984 | 2888.97 | 0.1560 | 4.16838 | 82.86 |
| 21.5501 | 1053.30 | 0.1920 | 4.12027 | 30.21 |
| 22.4104 | 443.85 | 0.1440 | 3.96400 | 12.73 |
| 23.0793 | 580.63 | 0.1920 | 3.85061 | 16.65 |
| 23.7993 | 1253.32 | 0.0960 | 3.73572 | 35.95 |
| 23.9411 | 1088.70 | 0.1680 | 3.71391 | 31.22 |
| 24.4051 | 1526.04 | 0.0600 | 3.64434 | 43.77 |
| 24.6201 | 375.83 | 0.3840 | 3.61301 | 10.78 |
| 4.9026 | 870.46 | 0.2880 | 357.266 | 24.96 |
| 25.7781 | 131.43 | 0.1680 | 3.45327 | 3.77 |
| 26.2003 | 146.75 | 0.2160 | 3.39857 | 4.21 |
| 26.8737 | 819.96 | 0.2640 | 3.31492 | 23.52 |
| 27.5478 | 826.93 | 0.0840 | 3.23531 | 23.72 |
| 28.0472 | 281.25 | 0.2400 | 3.17883 | 8.07 |
| 28.4482 | 274.28 | 0.1920 | 3.13492 | 7.87 |
| 28.8936 | 160.86 | 0.1920 | 3.08761 | 4.61 |
| 29.4369 | 567.10 | 0.2160 | 3.03184 | 16.26 |
| 29.9775 | 570.86 | 0.2400 | 2.97839 | 16.37 |
| 30.8403 | 332.96 | 0.2400 | 2.89700 | 9.55 |
| 31.5605 | 207.50 | 0.2640 | 2.83251 | 5.95 |
| 32.0676 | 138.20 | 0.1920 | 2.78887 | 3.96 |
| 32.3994 | 375.11 | 0.1440 | 2.76107 | 10.76 |
| 33.2585 | 289.29 | 0.4800 | 2.69168 | 8.30 |
| 33.9164 | 312.01 | 0.2400 | 2.64096 | 8.95 |
| 34.4433 | 90.29 | 0.1920 | 2.60175 | 2.59 |
| 35.4480 | 474.88 | 0.1200 | 2.53028 | 13.62 |
| 36.2603 | 498.08 | 0.0960 | 2.47544 | 14.29 |
| 36.9462 | 409.62 | 0.2400 | 2.43105 | 11.75 |
| 37.7103 | 254.45 | 0.1920 | 2.38352 | 7.30 |
| 38.3676 | 342.61 | 0.1200 | 2.34419 | 9.83 |
| 39.5559 | 64.04 | 0.3360 | 2.27646 | 1.84 |

### DSC/TGA

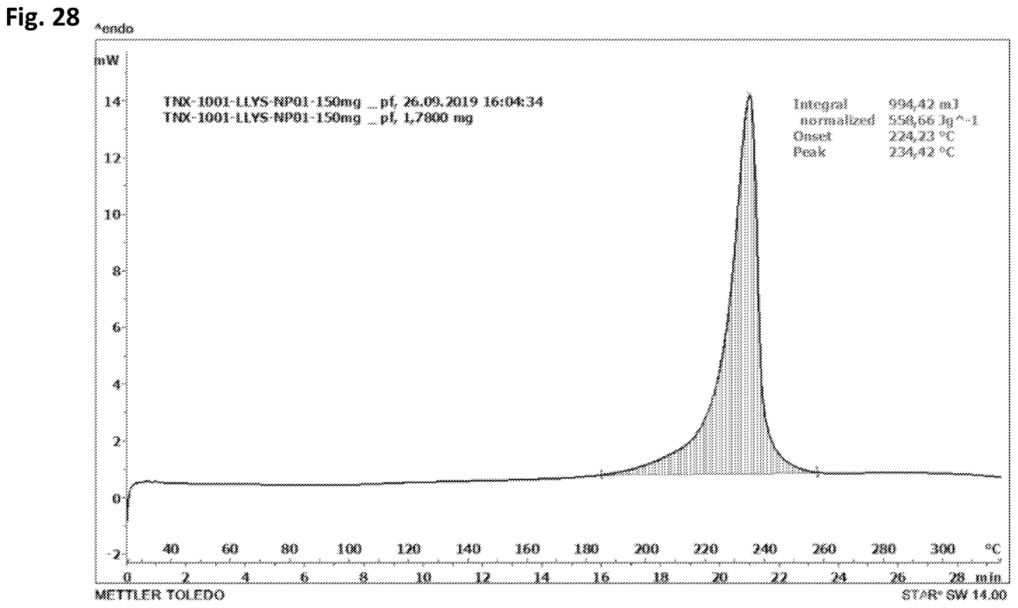

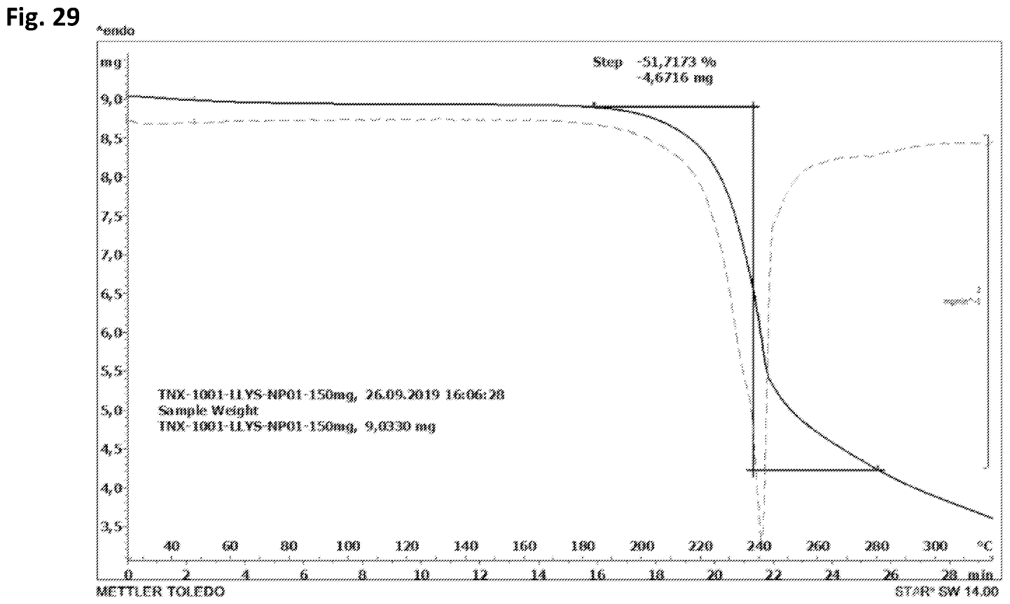

The DSC profile of sample TNX1001-LLYS-NP01 shows a single endothermic event at 234.4° C. (onset 224.2° C.) imputable to melting/degradation of the product (FIG. 28). The TGA profile is typical of an anhydrous compound decomposing above 200° C. (FIG. 29). EGA was consistent with the loss of carbonyl sulfide.

### FT-IR

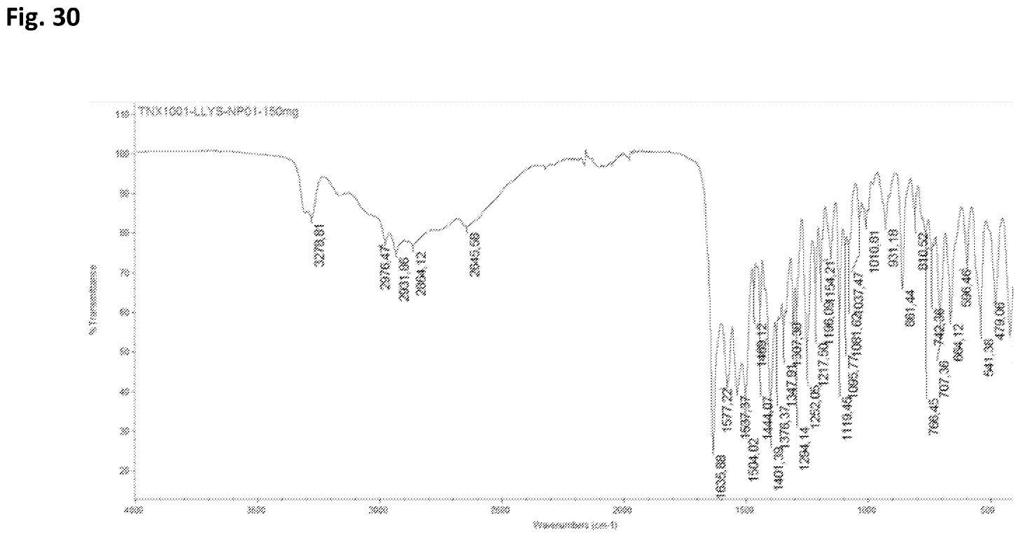

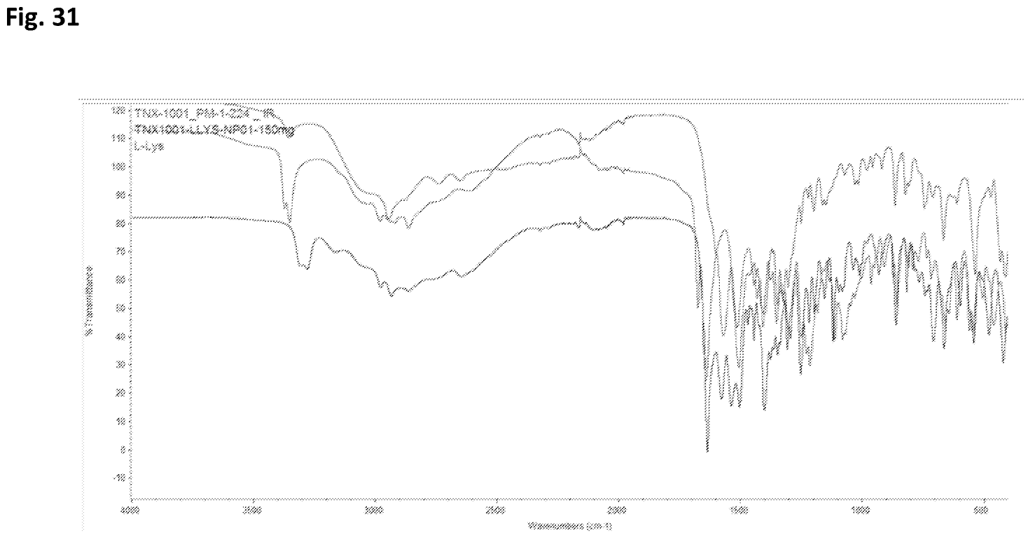

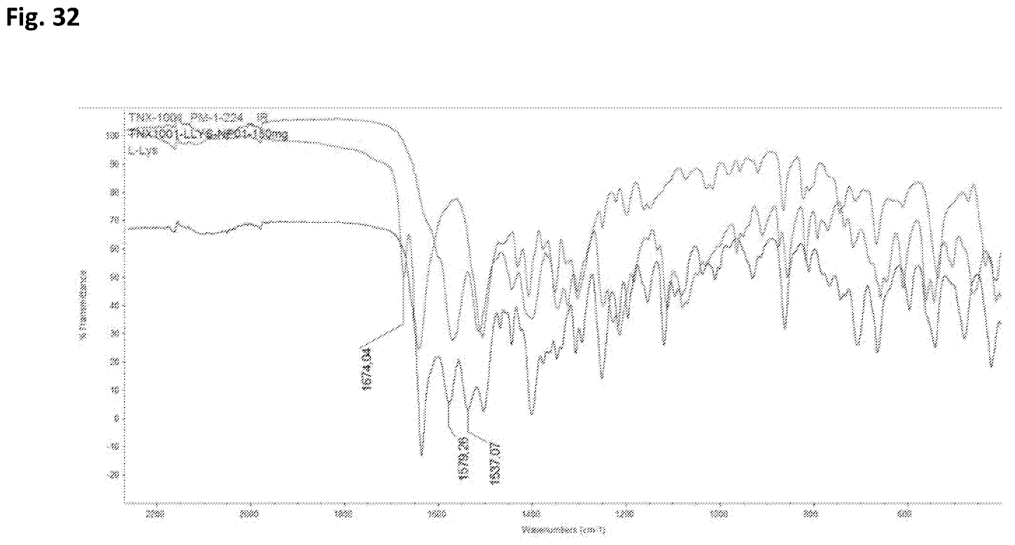

The FT-IR spectrum of sample TNX1001-LLYS-NP01 corresponds to FIG. 30. The corresponding FT-IR peak list is reported in Table 10 below. The comparison with the carbamathione starting material (TNX1001-PM-1-224) is reported in FIG. 31. The two spectra showed several differences. The most significant are the absence of the band at 1675 cm⁻¹ visible in the spectra of the carbamathione starting material and the presence of two new stretching band at 1579 cm⁻¹ imputable to carboxylate moiety of L-Lysine and at 1537 cm⁻¹, probably due to the formation of a new carboxylate moiety in the carbamathione (FIG. 32).

TABLE 10
FT-IR peak list of TNX1001-LLYS-NP01

| Position | Intensity |
|---|---|
| 421.20 | 53.555 |
| 479.06 | 62.687 |
| 541.38 | 60.102 |
| 596.46 | 71.992 |
| 664.12 | 58.273 |
| 707.36 | 60.599 |
| 742.36 | 75.195 |
| 766.45 | 79.593 |
| 810.52 | 84.189 |
| 861.44 | 65.974 |
| 931.18 | 82.201 |
| 1010.81 | 81.759 |
| 1037.47 | 83.700 |
| 1081.62 | 76.724 |
| 1095.77 | 76.419 |
| 1119.45 | 60.569 |
| 1154.21 | 74.413 |
| 1196.09 | 69.513 |
| 1217.50 | 66.345 |
| 1252.05 | 49.935 |
| 1294.14 | 61.299 |
| 1307.38 | 58.042 |
| 1347.91 | 56.222 |
| 1376.37 | 54.738 |
| 1401.39 | 38.343 |
| 1444.07 | 60.888 |
| 1469.12 | 66.102 |
| 1504.02 | 39.323 |
| 1537.37 | 39.754 |
| 1577.22 | 41.855 |
| 1635.88 | 24.943 |
| 2645.58 | 81.461 |
| 2864.12 | 76.612 |
| 2931.86 | 75.118 |
| 2976.47 | 77.913 |
| 3278.81 | 83.865 |

### ¹H NMR

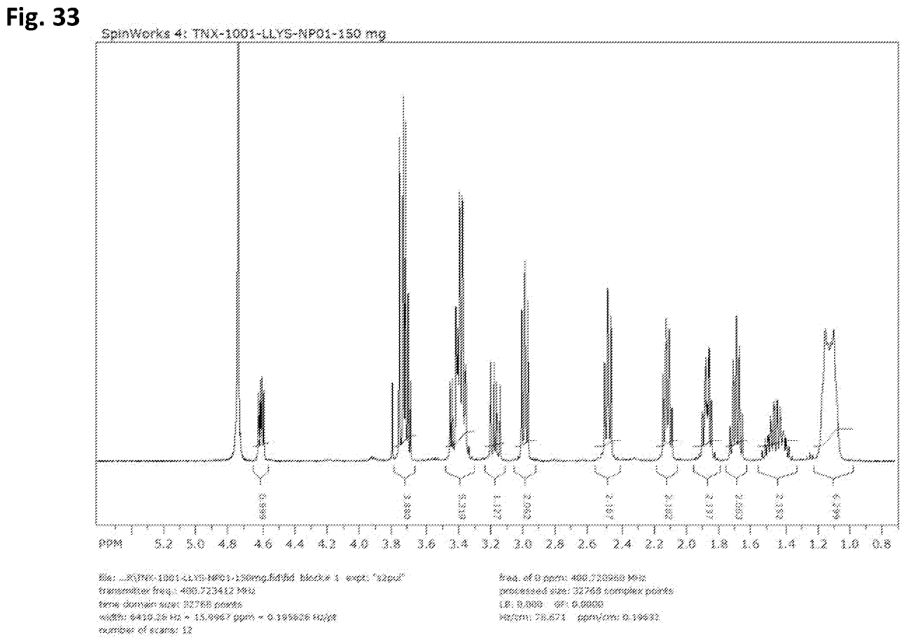

¹H-NMR confirmed the structural integrity of the carbamathione and the presence of L-Lysine in a 1:1 stoichiometric ratio. The NMR spectrum corresponds to FIG. 33. ¹H-NMR (D₂O, 400 MHz, temp: 25° C.); δ: 4.61 (dd, 1H, J=4.8, 8.4 Hz), 3.65-3.78 (m, 4H), 3.42 (dd, 1H, J=4.8, 14.4 Hz), 3.38 (quart., 2H, J=7.2 Hz), 3.37 (quart., 2H, J=7.2 Hz), 3.17 (dd, 1H, J=8.4, 14.4 Hz), 2.99 (t, 2H, J=7.6 Hz), 2.42-2.56 (m, 2H), 2.06-2.18 (m, 2H), 1.80-1.94 (m, 2H), 1.69 (quint., 2H, J=7.6 Hz), 1.32-1.56 (m, 2H), 1.01-1.22 (m, 6H).

### Characterization of TNX1001-LLYS-NP02

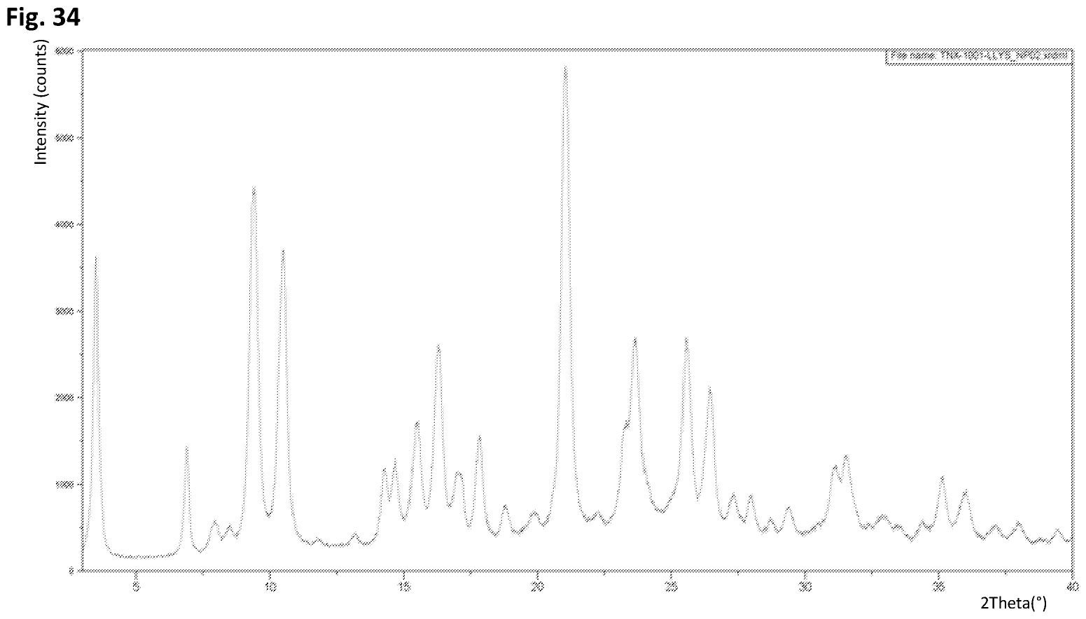

TNX1001-LLYS-NP02 was characterized by XRPD (see FIG. 34). The XRPD peaks are listed in Table 11 below.

TABLE 11
XRPD peak listing of TNX1001-LLYS-NP02

| Pos. | Height | FWHM | d-spacing | Rel. Int. |
|---|---|---|---|---|
| [° 2Th.] | [cts] | [° 2Th.] | [Å] | [%] |
| 3.4898 | 3347.69 | 0.1574 | 25.31855 | 61.28 |
| 6.8808 | 1240.59 | 0.1574 | 12.84675 | 22.71 |
| 7.9416 | 370.85 | 0.2755 | 11.13303 | 6.79 |
| 8.4864 | 318.83 | 0.1574 | 10.4194 | 5.84 |
| 9.3893 | 4203.94 | 0.2755 | 9.41941 | 76.96 |
| 10.4978 | 3465.46 | 0.3542 | 8.42718 | 63.44 |
| 11.8085 | 99.91 | 0.2362 | 7.49454 | 1.83 |
| 13.1836 | 149.14 | 0.2755 | 6.71579 | 2.73 |
| 14.2753 | 878.75 | 0.1968 | 6.20455 | 16.09 |
| 14.6759 | 932.93 | 0.1968 | 6.03609 | 17.08 |
| 15.4881 | 1399.05 | 0.2755 | 5.72133 | 25.61 |
| 16.299 | 2271.94 | 0.2755 | 5.43845 | 41.59 |
| 16.9589 | 797.17 | 0.1574 | 5.22829 | 14.59 |
| 17.1605 | 765.22 | 0.1574 | 5.16733 | 14.01 |
| 17.8328 | 1224.89 | 0.2362 | 4.97402 | 22.42 |
| 18.7908 | 422.68 | 0.2755 | 4.72253 | 7.74 |
| 19.9017 | 328.87 | 0.3149 | 4.46136 | 6.02 |
| 21.0389 | 5462.57 | 0.2755 | 4.2227 | 100 |
| 22.2459 | 331.71 | 0.1968 | 3.99625 | 6.07 |
| 23.2165 | 1259.95 | 0.1968 | 3.83133 | 23.07 |
| 23.6439 | 2313.7 | 0.2755 | 3.76303 | 42.36 |
| 25.5622 | 2285.71 | 0.2755 | 3.48483 | 41.84 |
| 26.4561 | 1706.93 | 0.2755 | 3.36907 | 31.25 |
| 27.31 | 491.05 | 0.2362 | 3.26565 | 8.99 |
| 27.9701 | 474.61 | 0.1968 | 3.19006 | 8.69 |
| 28.7106 | 201.75 | 0.1968 | 3.10944 | 3.69 |
| 29.3773 | 328.81 | 0.2755 | 3.04038 | 6.02 |
| 31.1156 | 806.16 | 0.2362 | 2.87437 | 14.76 |
| 31.5247 | 938.42 | 0.1968 | 2.838 | 17.18 |
| 32.9304 | 245.22 | 0.2755 | 2.72 | 4.49 |
| 33.6069 | 126.41 | 0.1574 | 2.66678 | 2.31 |
| 34.3804 | 210.22 | 0.1574 | 2.60853 | 3.85 |
| 35.1255 | 718.76 | 0.2362 | 2.55489 | 13.16 |
| 35.9765 | 543.83 | 0.3149 | 2.49638 | 9.96 |
| 37.0994 | 188.92 | 0.3149 | 2.42336 | 3.46 |
| 37.9876 | 218.71 | 0.2755 | 2.36872 | 4 |
| 38.818 | 42.34 | 0.2362 | 2.31994 | 0.78 |
| 39.4383 | 159.82 | 0.1968 | 2.28487 | 2.93 |

### Example 4. Hygroscopicity of TNX1001-LLYS NP01

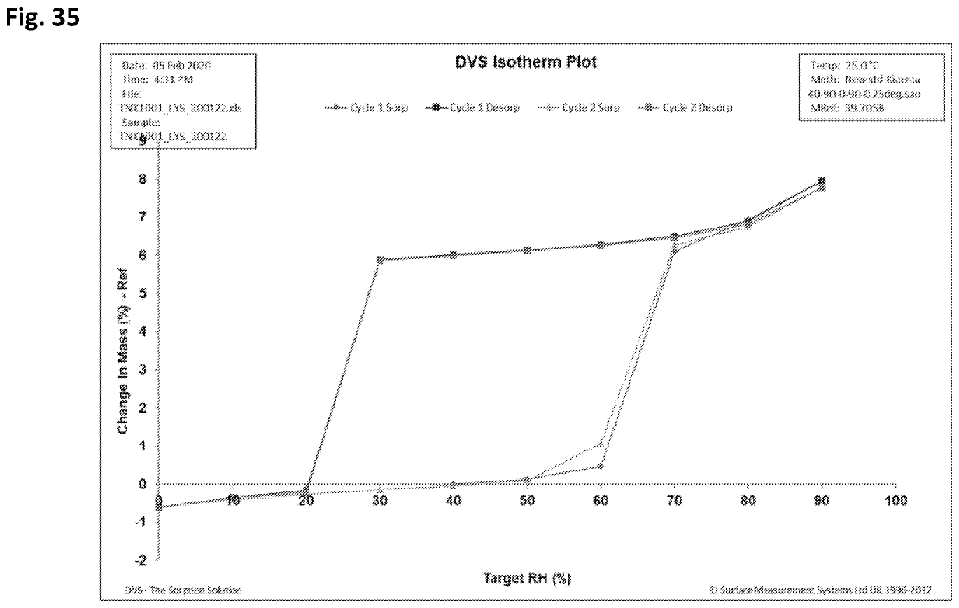

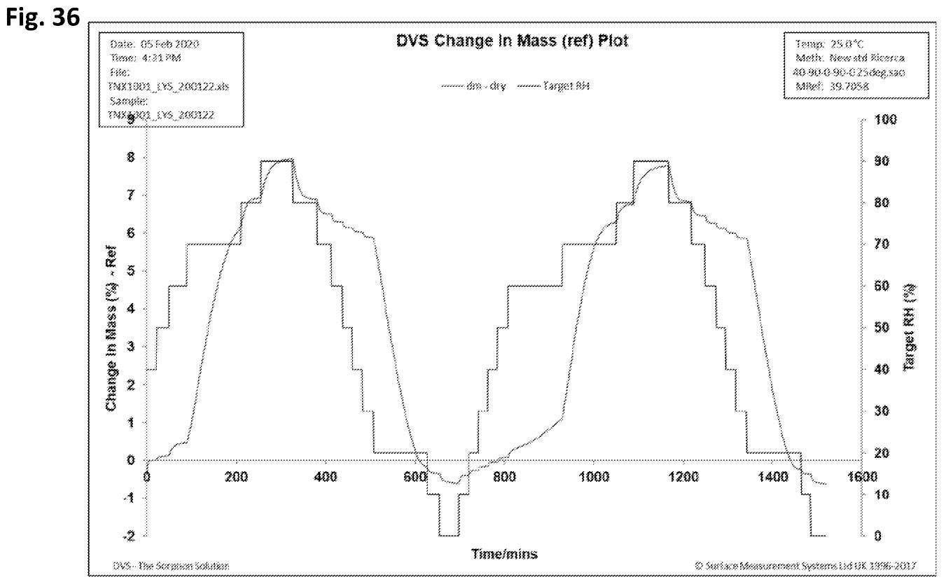

The anhydrous carbamathione lysine salt (TNX1001-LLYS-NP01) was subjected to dynamic vapor sorption (DVS) analysis (FIG. 35). The isotherm plot shows a sharp increase in mass in the sorption curves between 60% and 70% relative humidity (RH). Similarly, the desorption curves display a clear decrease in mass between 30% and 20% RH. This behavior is consistent with a compound forming a hydrate species. Additionally, based on the water uptake of approximately 6.1% w/w at 70% RH, the hydrated form of the salt is likely a dihydrate species (FIG. 36).

The sorption/desorption cycle was performed twice. The resulting sorption curves overlap almost perfectly, suggesting that the water uptake to form the hydrated species, and the water release to re-form the anhydrous species take place reversibly.

The sample was characterized by PXRD, ¹H NMR spectroscopy, and mass spec after DVS analysis, and confirmed the isolation of the anhydrous carbamathione lysine salt (TNX1001-LLYS NP01).

### Example 5. Stability Studies

Approximately 50 mg of the anhydrous carbamathione lysine salt was placed in a glass vial crimped with a PTFE/silicone septum and stored at the desired temperature and humidity for one month. Controlled humidity was realized employing saturated solutions of salts: NaCl for 75% RH at 40° C. and NaBr for 60% RH at 25° C. After storage, the samples were analyzed by XRPD analysis. Each stability test was performed in duplicate.

After one month of storage at 25° C. and 60% RH, no significant differences in the XRPD patterns were observed compared to the starting material, demonstrating that the anhydrous carbamathione lysine salt is stable under those conditions.

After one month of storage at 40° C. and 75% RH, no significant differences in the XRPD patterns were observed compared to the starting material, demonstrating that the anhydrous carbamathione lysine salt is stable under those conditions.

### Example 6. Solubility Studies

The dissolution profile of the anhydrous carbamathione lysine salt between 10-80° C. at three different pH values was assessed to extrapolate an approximate value for the solubility of TNX1001-LYS at 25° C.

Three different buffer solutions were prepared according to European Pharmacopoeia procedures (pH 1.2), or by diluting commercially available concentrate buffer solutions (pH 4.5 and 6.8).

Phosphate buffer at pH 6.8 was prepared by diluting a commercially available concentrate solution (Reagecon) with HPLC grade water. The final pH was adjusted using a 1 M NaOH solution.

Acetate buffer at pH 4.5 was prepared by diluting a commercially available concentrate solution (Reagecon) with HPLC grade water. The final pH was adjusted using concentrated acetic acid and a 1 M NaOH solution.

A buffer at pH 1.2 was prepared by mixing NaCl (0.2 M, 125 mL) and HCl (0.2 M, 212.5 mL) solutions followed by adjusting the volume to 500 mL. The pH was adjusted with a 1 M NaOH solution.

The determination of the dissolution temperature was performed in the automatic reactor system Crystal16. The system allows for careful control of the temperature and is equipped with a turbidimeter enabling the detection of the complete dissolution of the solid. The proper amount of compound was accurately weighted in a 1.5 mL vial equipped with a magnetic stirring bar. The selected buffer solution was pre-cooled in a refrigerator and the proper volume was added to the vial. The suspension was placed in the automatic reactor system pre-cooled at 10° C. and stirred at 600 rpm. The temperature was kept constant for 5 minutes to allow the system to equilibrate. The temperature was then increased at 0.5° C./min until a clear solution was obtained. For each pH, four solutions with increasing concentration were prepared and subjected to the same temperature program.

### Solubility at pH 6.8

The solubility of solutions having TNX1001-LLYS concentrations of 199 mg/mL, 222 mg/mL, 340 mg/mL and 397 mg/mL, respectively, at pH 6.8 were assessed. The two most dilute solutions turned clear during the equilibration period at 10° C. Dissolution temperatures of 24° C. and 33° C. were observed for the other two, more concentrated solutions.

In order to compare the solubility of the anhydrous carbamathione lysine salt with free carbamathione, the solubility of the free carbamathione was assessed at 25° C. by portion-wise addition of a known amount of solid to 5 mL of buffer. The solubility of free carbamathione was determined to be between 20 and 30 mg/mL, as 100 mg of free carbamathione dissolved completely in 5 mL of buffer, but a saturated solution was formed when a subsequent 50 mg aliquot of solid was added to the solution.

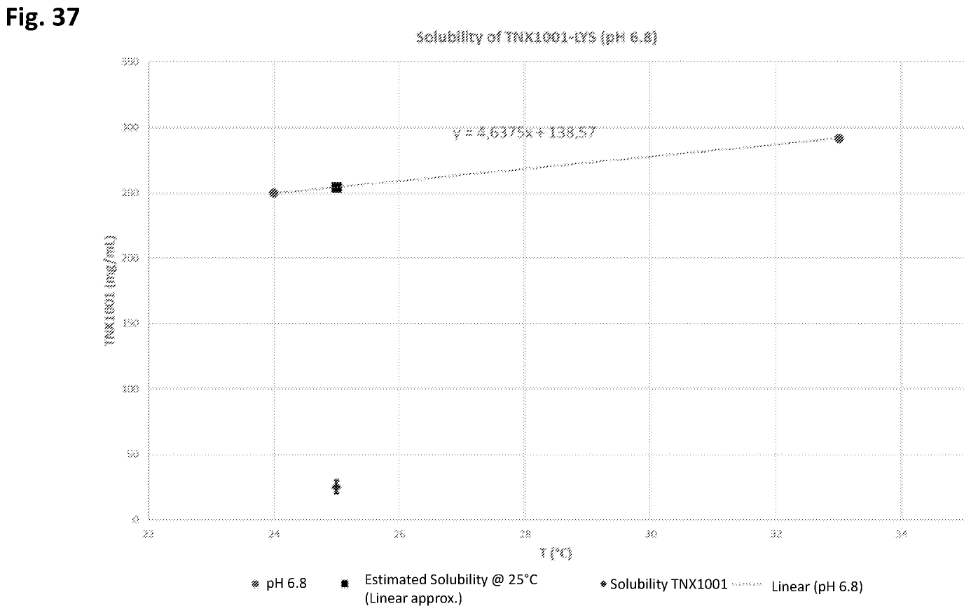

The solubility of TNX1001-LYS at 25° C. was estimated by a linear approximation considering the two experimental points available (FIG. 37). Although this is incorrect from a theoretical point of view, the closeness of the experimental value at 24° C. limited the error made by using this simple approximation.

The data are reported in Table 12. The comparison of the solubility of TNX1001-LLYs and free carbamathione demonstrates a solubility increase of approximately 10%.

TABLE 12
Dissolution data collected at pH 6.8

| | | Conc. | Conc. | |
|---|---|---|---|---|
| TNX1001- | Buffer pH | TNX1001- | TNX1001 | |
| LYS (mg) | 6.8 (mL) | LYS (mg/mL) | (mg/mL) | T_{diss.} (° C.) |
| 59.8 | 0.3 | 199 | 147 | <10 |
| 89.0 | 0.4 | 223 | 164 | <10 |
| 135.9 | 0.4 | 340 | 250 | 24 |
| 158.6 | 0.4 | 397 | 292 | 33 |

### Solubility at pH 4.5

The solubility of solutions having TNX1001-LLYS concentrations of 249 mg/mL, 299 mg/mL, 356 mg/mL and 401 mg/mL, respectively, at pH 4.5 were assessed. The most dilute solution turned clear during the equilibration period at 10° C. Dissolution temperatures of 16° C., 26° C. and 33° C. were observed for solutions having a TNX1001-LLYS concentration of 299 mg/mL, 356 mg/mL and 401 mg/mL, respectively

In order to compare the solubility of the anhydrous carbamathione lysine salt with free carbamathione, the solubility of the free carbamathione was assessed at 25° C. by portion-wise addition of a known amount of solid to 5 mL of buffer. The solubility of free carbamathione was determined to be between 10 and 20 mg/mL, as 50 mg of free carbamathione dissolved completely in 5 mL of buffer, but a saturated solution was formed when a subsequent 50 mg aliquot of solid was added to the solution.

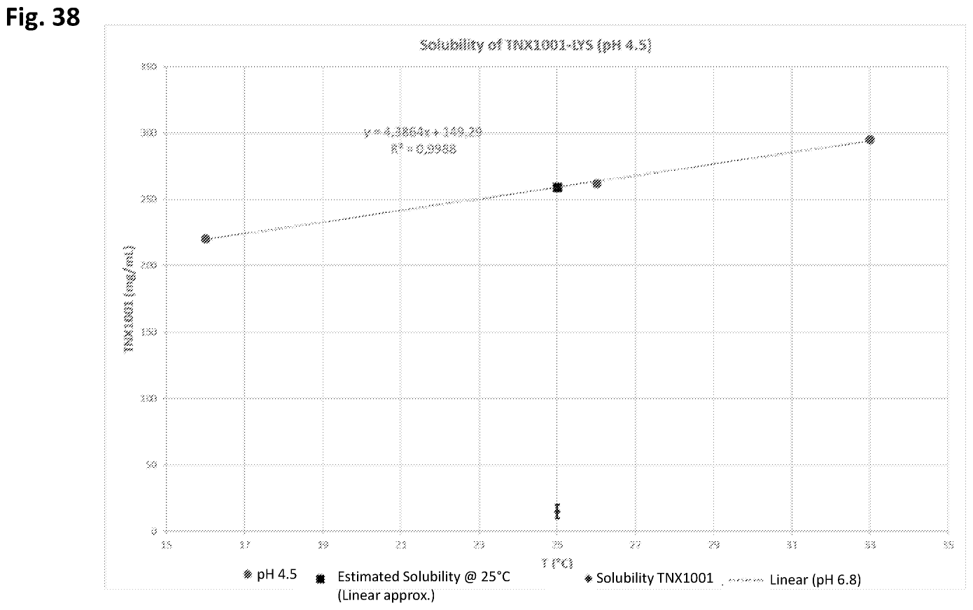

The solubility of TNX1001-LYS at 25° C. was estimated by a linear approximation considering the three experimental points available (FIG. 38). Although this is incorrect from a theoretical point of view, the closeness of the experimental value at 26° C. limited the error made by using this simple approximation.

The data are reported in Table 13. The comparison of the solubility of TNX1001-LLYS and free carbamathione demonstrates a solubility increase of approximately 17%.

TABLE 13
Dissolution data collected at pH 4.5

| | | Conc. | Conc. | |
|---|---|---|---|---|
| TNX1001- | Buffer pH | TNX1001- | TNX1001 | |
| LYS (mg) | 6.8 (mL) | LYS (mg/mL) | (mg/mL) | T_{diss.} (° C.) |
| 99.4 | 0.4 | 249 | 183 | <10 |
| 119.7 | 0.4 | 299 | 220 | 16 |
| 142.4 | 0.4 | 356 | 262 | 26 |
| 160.4 | 0.4 | 401 | 295 | 33 |

### Solubility at pH 1.2

The solubility of solutions having TNX1001-LLYS concentrations of 297 mg/mL, 349 mg/mL, 400 mg/mL and 455 mg/mL at pH 1.2 was attempted. However, under the experimental conditions tested, the lysine derivative was not stable and converted into the parent carbamathione, presumably due to lysine protonation by the HCl present in the buffer.

It was observed that the most dilute sample tested (297 mg/mL) almost completely dissolved at 10° C., but re-precipitation of free carbamathione rapidly occurred at the same temperature.

In the attempt to estimate a dissolution temperature, the suspensions were diluted to 1.5 mL and heated at 0.5° C./min until 80° C., but complete dissolution did not occur. Increasing the temperature to 90° C. the formation of a clear solution was observed in every case, but reliable data to build a solubility curve could not be collected.

After solid dissolution, the clear solutions were allowed to cool spontaneously to RT. XRPD analysis of the precipitated solid was performed, confirming that the precipitation of free carbamathione occurred in every case.

The experiment is summarized in Table 14 below.

TABLE 14
Dissolution data collected at pH 1.2

| | | Conc. | |
|---|---|---|---|
| TNX1001- | Buffer pH | TNX1001-LYS | |
| LYS (mg) | 6.8 (mL)¹ | (mg/mL)² | T_{diss.} (° C.) |
| 118.9 | 0.4 (1.5) | 297 (79) | Dissolve and re-precipitate |
| 139.4 | 0.4 (1.5) | 349 (93) | Dissolves above 80° C. |
| | | | after dilution |
| 160.0 | 0.4 (1.5) | 400 (107) | Dissolves above 80° C. |
| | | | after dilution |
| 182.1 | 0.4 (1.5) | 455 (121) | Dissolves above 80° C. |
| | | | after dilution |

¹The value in parentheses refers to the final volume after dilution.
²The value in parentheses refers to the concentration after dilution.

The results of the estimated solubility data for TNX1001-LYS and the comparison with the parent carbamathione are summarized in Table 15 below. The collected data show an increase of the solubility of the lysine derivative of approximately one order of magnitude compared to the parent carbamathione at pH 6.8 and 4.5. The determination of the solubility at pH 1.2 was not possible because after an initial fast dissolution of the solid, the re-precipitation of free carbamarhione took place rapidly.

TABLE 15
Summary of estimated solubility data for free carbamathione
(TNX1001) at 25° C. and the carbamathione lysine salt
(TNX1001-LLYS) (the solubility of TNX1001-LYS) is
expressed as equivalent amount of TNX1001 dissolved.

| | Solubility TNX1001 | Solubility TNX1001-LYS |
|---|---|---|
| pH | (mg/mL) | (mg/mL of TNX1001) |
| 6.8 | 20-30 | 255 |
| 4.5 | 10-20 | 299 |
| 1.2 | 25-33 | Not stable. TNX1001 |
| | | precipitates |

### Example 7. Polymorph Screening

The preparation of TNX1001-LLYS-NP01 and TNX1001-LLYS-NP02 is scaled up to produce a batch (50 g approximately) for use in polymorph screening investigations.

### Solvent Solubility Screening

The impact of different solvents on the polymorphism of TNX1001-LLYS-NP01 and TNX1001-LLYS-NP02 is assessed. Initially, the visible solubility of TNX1001-LLYS-NP01 and TNX1001-LLYS-NP02 is independently assessed according to the procedure described in the European Pharmacopeia. Classification of the solvents according to the visual solubility of TNX1001-LLYS-NP01 and TNX1001-LLYS-NP02 is determined based on the groups described in Table 16.

TABLE 16
Solubility rages description

| | | Parts of solvent needed | Solubility |
|---|---|---|---|
| Descriptive Terms | Abbreviation | for 1 part solute | (mg/mL) |
| Very soluble | VS | <1 | >1000 |
| Freely Soluble | FS | 1-10 | 100-1000 |
| Soluble | S | 10-30 | 33-100 |
| Sparingly Soluble | SS | 30-100 | 10-33 |
| Slightly Soluble | VSS | 100-1000 | 1-10 |
| Very Slightly | | 1000-10000 | 1-0.1 |
| soluble | | | |
| Insoluble | INS | >1000 | <0.1 |

### Evaporation

TNX1001-LLYS-NP01 and TNX1001-LLYS-NP02 are assessed independently in each of the solvents. 50 mg of sample is dissolved in 5 mL of each solvent. The solution is stirred for approximately 60 minutes. The solution is filtered with a Whatman 0.45 μm filter and left to evaporate. The experiment is performed in solvents where the compound is very soluble, freely soluble, soluble and sparingly soluble. The evaporation conditions range from low temperature (4-10° C.), room temperature (17-25° C.), high temperature (40-60° C.), and under 1 atm, or reduced pressure (10⁻² atm).

A set of binary solvent mixtures is defined for further evaporation experiments based on solubility data, solvent miscibility and the outcome of the single solvent evaporation experiments.

For samples that are classified as sparingly soluble, the evaporation of saturated solutions is performed as follows: 3 mL of a saturated solution is prepared by dissolving the sample (max 300 mg) at room temperature. The solution is filtered with Whatman 0.45 μm filter and left to evaporate. The resulting solid is collected and analyzed by XRPD.

### Slurry Experiments

When TNX1001-LLYS-NP01 or TNX1001-LLYS-NP02 has a solubility in a selected solvent that is ≤10 g/L, a slurry experiment is performed. The salt (30-50 mg) is suspended in 600-1500 μL of a single solvent and allowed to stir at approximately 350 rpm under varying conditions. Examples of conditions that are used in this experiment are as follows:
3 days at room temperature (25° C.)
3 days at high temperature (50° C.)
15 days at room temperature (25° C.)
3 days at variable temperature as described
From 10° C. to 50° C. at 20° C./hours
3 hours at 50° C.
From 50° C. to 10° C. at −20° C./hours
3 hours at 10° C.
From 10° C. to 50° C. at 10° C./hours
3 hours at 50° C.
From 50° C. to 10° C. at −10° C./hours
3 hours at 10° C.
From 10° C. to 50° C. at 5° C./hours
3 hours at 50° C.
From 50° C. to 10° C. at −5° C./hours
3 hours at 10° C.
From 10° C. to 25° C. at 10° C./hours
24 hours at 25° C.

The suspension is recovered, filtered under vacuum and analyzed by XRPD.

The slurry experiment is also performed in a mixture of solvents. The salt (40 mg) is suspended in 4 mL of a pre-prepared mixture of solvents, and left to stir at approximately 350 rpm. The slurry is allowed to stir for an extended period of time, and at varying temperature. As an example, the slurry is allowed to stir for 7 days at room temperature (25° C.) or for 3 days at high temperature (50° C.). The suspension is recovered, and filtered under vacuum. The resulting solid is analyzed XRPD.

### Precipitation

The solvents for the precipitation experiments are selected based on solubility data of TNX1001-LLYS-NP01 and TNX1001-LLYS-NP02 in varying solvents. Methods used in precipitation experiments include, by way of example, precipitation by anti-solvent addition, or precipitation by gradient temperature.

For precipitation by anti-solvent addition, the starting material (either TNX1001-LLYS-NP01 or TNX1001-LLYS-NP02) is suspended in a solvent to obtain a suspension at room temperature. The suspension is left stirring overnight followed by filtration with a Whatman filter (0.45 μm) to obtain a clear solution. The mixture of the clear solution with the anti-solvent is performed in any one of the following ways:
anti-solvent is added dropwise to the solution under magnetic stirring at room temperature (PAD);
the solution is added dropwise to the anti-solvent under magnet stirring at room temperature (PAI);
the saturated solution is exposed to vapors of a low-boiling anti-solvent at room temperature for 7-10 days (PASD).

The resulting precipitate is filtered under vacuum and analyzed by XRPD. If no precipitate forms, the solution is stored at low temperature (8° C.) for 24 hours. If no precipitate occurs, the solution is left at −20° C. for 24 hours. The resulting solid is collected and analyzed by XRPD.

For precipitation experiments by gradient temperature, a suspension of TNX1001-LLYS-NP01 or TNX1001-LLYS-NP02 is heated to 100° C. (as allowed by the solvents boiling point) to induce complete dissolution. The solution is then cooled. The cooling process can be carried out according to a variety of methods. For example, the hot solution is:
cooled down to 10° C. applying a ramp of 0.5° C./min and then the precipitate is recovered under vacuum after approximately 30 minutes from the end of the ramp (PSS);
cooled down at 10° C. by crash cooling in an ice bath, followed by precipitate recovery under vacuum after 5-10 minutes from the precipitation event (PSF);
cooled down at 25° C., followed by precipitate recovery under vacuum after 5-10 minutes from the precipitation event (PPT_RT).

The resulting precipitate is filtered under vacuum and analyzed by XRPD. If no precipitate forms, the solution is stored at low temperature (8° C.) for 24 hours. If no precipitate occurs, the solution is left at −20° C. for 24 hours. The resulting solids are collected and analyzed by XRPD.

### Full Physical Characterization of New Forms

For all new crystalline phases, the reproducibility of the crystallization procedure is performed. A preliminary assessment of their stability is carried out under varying conditions. For example, the sample is left at room temperature, pressure and relative humidity conditions. Additionally, the stability of the sample is assessed after 7 days of storage in a sealed vial at room temperature. For phases that show sufficient stability, a suitable amount of the sample is characterized via methods that are well known in the art. For example, XRPD, FT-IR/FT-Raman, DSC, TGA-EGA, DVS, DF, XRPD after grinding, and/or kneading and/or after storage at 25° C./60% RH/7 days, and/or after storage at 60° C./75% RH/3 days. The integrity of the molecule is assessed by re-crystallization or other suitable procedures and the interconversion diagram for the isolated forms is used to identify the most stable crystalline form.

1. A salt form of S—(N, N-diethylcarbamoyl)glutathione, wherein the salt is selected from the group consisting of an acetate salt, an adipate salt, an ascorbate salt, a benzoate salt, a camphorate salt, a citrate salt, a fumarate salt, a glutarate salt, a glycolate salt, a hydrochloride salt, a tartrate salt, a malate salt, a maleate salt, a methanesulfonate salt, an ethanedisulfonate salt, an ethanesulfonate salt, a naphthalenesulfonate salt, an oxalate salt, a phosphate salt, a sulfate salt, a sorbate salt, a benzenesulfonate, a cyclamate salt, succinate salt, a toluenesulfonate salt, an arginine salt, a lysine salt, a deanol salt, a choline salt, a sodium salt, a potassium salt, a diethylammonium salt, a meglumine salt, a pyridoxine salt, a tris(hydroxymethyl)ammonium salt a N-cyclohexylsulfamate salt, a camphor-10-sulfonate salt, a naphthalenedisulfonate salt, and a quinaldate salt, or its solvates, polymorphs, hydrates or mixtures thereof.

2. The salt form according to claim 1, wherein the salt is a lysine salt or a solvate, polymorph, hydrate or mixture thereof.

3. The salt form according to claim 2, characterized by:
(i) a ¹H-NMR spectrum having peaks at about 4.61, about 3.65-3.78, about 3.42, about 3.38, about 3.37, about 3.17, about 2.99, about 2.42-2.56, about 2.06-2.15, about 1.80-1.94, about 1.69, about 1.32-1.56 and about 1.01-1.22 ppm when recorded in D₂O on a 400 MHz instrument; or
(ii) a XRPD pattern having peaks at about 3.6959, about 9.4909, about 10.6341, about 14.9275, about 18.0999, about 18.9789, about 19.5979, about 20.0613, about 20.1184, about 20.8543, about 21.5501, about 23.7993, about 23.9411, and about 24.4051 degrees 2Theta when measured using a Cu X-ray source, 1.54 Angstroms, tube voltage 40 kV and tube output 15 mA.

4. The salt form according to claim 2 characterized by a XRPD pattern having peaks at about 3.4898, about 6.8808, about 9.3893, about 10.4978, 15.4881, about 16.299, about 17.8328, 21.0389, about 23.2165, about 25.5622, about 26.4561, about 31.5247 degrees 2Theta when measured using a Cu X-ray source, 1.54 Angstroms, tube voltage 40 kV and tube output 15 mA.

5. The salt form according to claim 2, wherein the solubility of the salt form is between 5% and 90% higher than free S—(N, N-diethylcarbamoyl)glutathione.

6. The salt form according to claim 5, wherein the solubility of the salt form is between 5% and 20% higher than free S—(N, N-diethylcarbamoyl)glutathione.

7. The salt form according to claim 1, wherein the salt form is crystalline, co-crystalline, semi-crystalline or an amorphous powder.

8. A pharmaceutical composition comprising:
(i) a therapeutically effective amount of a salt form according to claim 1, wherein the salt form is crystalline, co-crystalline, semi-crystalline or an amorphous powder, or its solvates, polymorphs, hydrates or mixtures thereof; and
(ii) at least one pharmaceutically acceptable carrier.

9. (canceled)

10. The pharmaceutical composition according to claim 8, wherein the composition is formulated for oral administration, sublingual administration, intranasal administration, transdermal administration, subcutaneous administration, intramuscular administration, intraperitoneal administration, intravenous administration, conjunctival administration, intrathecal administration, by inhalation into the lung or rectal administration.

11. The pharmaceutical composition of claim 10, wherein the composition is formulated for oral administration.

12. The pharmaceutical composition according to claim 8, wherein the pharmaceutically acceptable carrier is a liquid diluent.

13. The pharmaceutical composition according to claim 8, wherein the pharmaceutically acceptable carrier is selected from the group consisting of tablets, scored tablets, coated tablets, orally dissolving tablets, thin films, caplets, hard capsules, soft gelatin capsules, troches, dragees, dispersions, suspensions, aqueous solutions, liposomes, patches, and sustained release formulations.

14. The pharmaceutical composition according to claim 8, further comprising suspending agents, emulsifying agents, non-aqueous vehicles, flavorings, colorings, antimicrobial agents, preservatives, or agents that form eutectics with the salt form of claim 1.

15. A method of preventing or treating a glutamate-related disorder in a subject in need thereof or at risk thereof, comprising administering to said subject a therapeutically effective amount of a composition according to claim 8.

16. The method according to claim 15, wherein the subject is a human.

17. The method according to claim 15, wherein the glutamate-related disorder is selected from the group consisting of Huntington's disease, Alzheimer's disease, Parkinson's disease, acquired immunodeficiency syndrome (AIDS) neuropathy, epilepsy, an eating disorder, a sleep disorder, nicotine addiction, cerebral ischemia, familial Amyotrophic Lateral Sclerosis (ALS), gambling disorder, mood symptoms relating to addiction withdrawal, neurodegenerative diseases associated with thiamine deficiency, Wemicke-Korsakoff syndrome, cerebral beriberi, Machado-Joseph disease, Soshin disease, and related diseases, anxiety, glutamate related convulsions, hepatic encephalopathy, neuropathic pain, domoic acid poisoning, hypoxia, anoxia, mechanical trauma to the nervous system, hypertension, alcohol withdrawal seizures, alcohol addiction, alcohol craving, cardiovascular ischemia, oxygen convulsions, hypoglycemia, Creutzfeldt-Jakob disease, cocaine addiction, noise induced hearing loss, nicotine addiction, heroin addiction, addiction to opioids, cyanide-induced apoptosis, schizophrenia, bipolar disorder, peripheral neuropathy associated with diabetes and non-ketonic hyperglycinemia.

18. The method according to claim 17, wherein the glutamate-related disorder is an alcohol use disorder.

19. The method according to claim 18, wherein the alcohol use disorder is selected from the group consisting of alcohol addiction, alcohol abuse, alcohol dependence, alcohol withdrawal seizures and alcohol craving.

20. The method according to claim 15, wherein the salt form of S—(N, N-diethylcarbamoyl) glutathione is administered at a concentration of from 0.5 mg/kg to 500 mg/kg.

21. The method according to claim 15, wherein the salt form of S—(N, N-diethylcarbamoyl) glutathione achieves a plasma level in the subject, after administration, of from 2 to 100 nmol/L.
